(19) United States
(12) Patent Application Publication
(10) Pub. No.: US 2025/0264465 A1
(43) Pub. Date: Aug. 21, 2025

(54) System, Apparatus, and Method for Detecting Single Molecular Binding Event Strength with Application to Protein Conformation Monitoring and Spatial-Temporal Multiomics Mapping in In Vitro and In Vivo Environments

(71) Applicant: Poynter Technologies, Inc., Portland, OR (US)

(72) Inventors: Devin Thomas Wiley, Portland, OR (US); David Nathan Shykind, Buxton, OR (US); Steven Bowers, Oak Park, CA (US); Jane Ni, Portland, OR (US)

(21) Appl. No.: 19/057,819

(22) Filed: Feb. 19, 2025

Related U.S. Application Data
(60) Provisional application No. 63/555,276, filed on Feb. 19, 2024.

(51) Int. Cl. G01N 33/557; G01N 27/22; G01N 33/53

(52) U.S. Cl. CPC G01N 33/557; G01N 33/5302; G01N 27/227

(57) ABSTRACT

An apparatus for detecting and communicating measurements related to the occurrence, magnitude, and duration of one or more binding events between a targeting agent and a target is disclosed. The apparatus may comprise a sensing subsystem with at least one targeting agent and electrically conductive linkers to enable detection of a target within a chemical or biological environment or sample. A deciding subsystem facilitates the identification of single binding events and their duration. Signaling circuitry processes input signals and transmits binding event-related data to a user device. The apparatus may be housed with additional components to measure binding characteristics of drug candidates, enabling precise, efficient, and cost-effective testing, screening, and validation of drug candidates. Alternatively, the apparatus may be configured and used to analyze spatial and temporal characteristics at the genomic, protein, and post-translational modification levels in cells, tissues, and organs in both in vitro and in vivo contexts.

### CROSS-REFERENCE TO RELATED APPLICATIONS

This application claims the benefit of U.S. Provisional Application No. 63/555,276, filed on Feb. 19, 2024, entitled “System, Apparatus, and Method for Determining Pharmacokinetics and Spatial Genomics Qualities of a Target in an In Vitro or In Vivo Environment,” which is hereby incorporated by reference in its entirety.

### FIELD OF THE INVENTION

The present invention relates to bioanalytic, diagnostic, and/or therapeutic devices, and methods of use thereof, that interact with, sense, and identify chemical and biological materials and can detect and communicate measurements indicative of the occurrence and duration of one or more binding events between a targeting agent and a target, as well as help determine the genotype and phenotype of living systems, including information about a gene and gene-product (i.e., protein and protein modification) 3-dimensional conformation and specific location over time.

### BACKGROUND OF THE INVENTION

The background description provided herein is for the purpose of generally presenting the context of the disclosure. Work of the presently named inventors, to the extent it is described in this background section and aspects of the description that may not otherwise qualify as prior art at the time of filing, are neither expressly nor impliedly admitted as prior art against the present disclosure.

Testing at each stage of drug development is critical to conduct an efficient and cost-effective process that ensures optimal efficacy, bioavailability, exposure, safety, dosage, and appropriate route of administration of a drug of interest, amongst other parameters. Drugs often initiate their effects by binding to a biomolecular target. Therefore, a drug's “binding affinity” or “binding strength” to a target plays an important role in researching and understanding the drug's efficacy, safety, and duration of action.

To induce its effect, a drug is often designed to target molecular interactions associated with specific diseases to either block or mimic the interaction. Therefore in pharmaceutical research and drug discovery, determining and optimizing binding affinity (or avidity), denoted by the equilibrium dissociation constant, K_{D}, is critically important for developing new drugs and treatment options.

Binding affinity refers generally to the strength of the binding interaction between a biomolecule and its target (or ligand). Information about a drug's binding affinity to its target can determine its selectivity, potency, and optimal dosage, parameters that impact a drug's efficacy and safety.

As is understood in the art, higher affinity is usually desired, as lower concentrations of a therapeutic are needed to achieve the desired effect, which typically reduces the risk of undesirable off-target effects. Affinity is typically measured and reported using the thermodynamic equilibrium dissociation constant, K_{D}. As is understood in the art, the smaller the K_{D} value, the greater the binding affinity.

Interactions between biomolecules in solution are dynamic processes. These processes are described by standard chemical kinetics equations, with the kinetic rate of association or binding given by a forward rate constant (“kₒₙ”) and the reverse reaction by a disassociation rate constant (“k_{off}”). The ratio of the forward (binding) rate constant to the dissociation rate constant (kₒₙ/K_{off}) at equilibrium determines the fraction of occupied sites (which have bound ligands), and conversely, the fraction of unbound or unoccupied binding sites at equilibrium is described by the inverse ratio (K_{off}/kₒₙ) also referred to as the dissociation constant or K_{D}. The free energy of the binding reaction may be related to K_{D} via the Gibbs formula DG=DGₒ+R*T*ln(K_{D}), where R is the ideal gas constant and T is the absolute temperate (° K). The binding energy may be further used to determine details of the reaction, such as residence time. A strongly bound ligand will necessarily have a small K_{D} and vice versa. “High-affinity” binding sites will perforce have a small K_{D}. Quantifying a particular drug's kₒₙ and k_{off} rate constants is, therefore, highly relevant to understanding the drug's overall binding strength to its desired target and its potential therapeutic potency. In particular, the time a drug spends in contact with its biological target (residence time, calculated by either t_{off}-tₒₙ or 1/k_{off}) is considered a key parameter for drug optimization as it is understood in the art to correlate to drug efficacy in vivo.

Optimizing K_{D} is a crucial step during the drug discovery “lead optimization” process as it decreases failure rates at the more advanced and much more costly “drug candidate” and clinical trial stages. Accordingly, there is a need for high-throughput and high-accuracy methods to profile the binding strength of hundreds to thousands of compounds to a therapeutic target through the measurement of K_{D} values.

Currently available binding assays rely on methods including label-free, optical (fluorescence-based), radioactive, or enzyme-linked detection techniques. These current technologies generally provide some bulk measure of the interactions between two populations of biomolecules, such as protein binding or oligonucleotide annealing. Consequently, these technologies yield a single measure of binding strength (K_{D}) that describes the ensemble average of one molecular population's binding strength to another. Relative to the invention described herein, these methods are unable to detect single binding events and are insensitive to detecting potential subpopulations of molecules that may have different binding strength properties from the bulk (for instance, by a subpopulation of drug that has a different molecular conformation owing to the synthetic route or degradation, or a diverse population of binding molecules or sites). Further relative to the invention described herein, to varying degrees, contemporary binding assay methods are insensitive and lack precision, are time-consuming (low throughput), excessively complex, and may require expensive instrumentation.

Therefore, a need exists for a system (apparatus or device) and method that can measure binding affinity characteristics at the level of single molecular interactions, yield quantifiable results (including repeatable results measured over a period of time), have high throughput, be less complex, and be more cost-effective.

Further, a need exists for a system (apparatus or device) and method that can provide scientifically and clinically relevant information on how amino acid sequences in proteins can dictate their 3-D conformation in different physiologic and pathologic environments, known in the art as the “protein folding problem.” As is understood in the art, proteins consist of chains of amino acids that spontaneously fold, in a process called protein folding, to form the proteins' three-dimensional (3-D) structures (conformations). This 3-D structure is crucial to the biological function of the protein and can be altered to a non-functioning or even toxic form in a pathological environment. This “protein folding problem” involves understanding the interatomic forces that determine the folded stable structure, how the native structure of a protein can be predicted from its amino acid sequence, the mechanism, and pathway through which a protein can reach its final folded state, and how the conformation of a protein may be changed in a pathologic state (for example in a hypoxic and acidic environment within a tumor or other ischemic tissue).

Protein structures are currently determined experimentally by means of techniques such as X-ray crystallography, cryo-electron microscopy, and nuclear magnetic resonance, techniques which are both expensive and time-consuming. Such efforts have identified the structures of about 170,000 proteins over the last 60 years, while there are over 200 million known proteins across all life forms. Therefore, a need exists for a system (apparatus or device) and method that can cost-effectively and rapidly provide data related to protein structure in relation to an amino-acid sequence and monitor changes to protein conformation when exposed to a pathologic or other non-physiologic environment.

A need further exists for a system (apparatus or device) and method that can provide scientifically and clinically relevant information about a target's spatial and temporal characteristics at the genomic, protein, and post-translational modification level within cells, tissues, and organs (described herein to encompass the detection of all forms of genomic, transcriptomic, and proteomic biomolecules by the term “spatial-temporal multiomics” or “STM”). Beyond the measurement of binding kinetics, affinity, and avidity (binding strength through multivalent molecular binding), STM is essential to understanding within a positional and temporal context the relationships amongst genome organization, differential gene expression, epigenetic modification, gene transcription, and translation, and the resulting proteome including post-translational modifications.

Currently, in spatial genomics, it is understood in the art that ascertaining the transcriptome of a cell, tissue, or organ with spatial information entails removing and slicing tissue into thin sections and then anchoring sections of that thinly sliced tissue to a physical array that tags mRNA with spatial information. RNA is then sequenced using techniques that are understood in the art. Relative to the invention described herein, these current methods are unable to resolve both spatial and temporal information of multiple types of biomolecules (for example, mRNA, proteins, and post-translational modifications) across a variety of samples (including cells, tissues, and organs), both in an in vitro and in vivo context. Unfortunately, it is further understood in the art that cells express fewer genes when removed from their microenvironment and processed using current methods. Thus, data obtained in this manner does not always provide an accurate picture of the cell as it would exist in its “native” (in vivo) microenvironment.

Therefore, there is a need for a system (apparatus or device) and method that may accurately analyze the STM qualities of a cell, tissue or organ, in both an in vitro or in vivo context, including within living (non-fixed) systems. It is against this background that the present invention has been developed.

### BRIEF SUMMARY OF THE INVENTION

Disclosed herein are examples of a binding event sensor that can comprise a sensing-deciding device (“SDD”) that detects the occurrence and/or measures the magnitude of a binding event between a targeting agent and a target based on a change of impedance sensed by the device. Aspects that may be included as part of the present invention are described in greater detail below.

For example, the inventors disclose an SDD that measures clock data associated with a binding event.

The inventors also disclose an SDD that outputs or transmits data indicating the existence, magnitude, and/or duration of the electrical signal generated by one or more targeting agent-target binding event(s) to a receiver or transceiver coupled to a computing device.

Further still, disclosed herein is an SDD that may be contained within a casing, enclosure, housing, or structure to enable or facilitate device handling, preparation, and operation by a user.

Also disclosed herein is an SDD that may include a casing, enclosure, housing, or structure having a surface that contains or encloses supporting logic and other integrated circuitry, at least one electrically conductive pad positioned on, proximate to, or within the casing, enclosure, housing, or structure surface and connected to the logic and other integrated circuitry and to at least one targeting agent capable of interacting with a target, wherein in response to target interaction, the logic circuitry allows signaling circuitry to transmit information to a receiver or transceiver, either by wire or wirelessly, regarding binding between one or more targeting agents and one or more targets.

Further still, disclosed herein is an SDD that may include at least one electrically conductive pad positioned on, proximate to, or within the casing, enclosure, or housing surface and connected to the logic circuitry and to at least one targeting agent capable of interacting with a target.

Also disclosed herein is an SDD that may include at least one electrically conductive pad, wherein each electrically conductive pad is addressable and associated with a unique identifier, such that binding event-related data associated with a particular electrically conductive pad (and therefore, its associated targeting agent) may be correlated to a particular targeting agent for meaningful output or display to a user.

Further still, the inventors disclose an SDD that may include one or more targeting agents and signaling circuitry that transmits information to a receiver or transceiver over a wire, wirelessly, or a combination thereof, regarding binding between one or more targeting agents and one or more targets.

Still further, the inventors disclose an SDD that may include one or more targeting agents and signaling circuitry to transmit, over a wire, wirelessly (e.g., through RF signaling), or a combination thereof, information to a receiver or transceiver regarding binding between one or more targeting agents and one or more targets (i.e., binding events) based on a change in impedance (in comparison to a reference provided by a reference agent) that exceeds a minimum threshold.

Also disclosed is an SDD whereby a change in impedance caused by a binding event between a targeting agent and target is connected electrically to amplifier circuitry that facilitates the detection of the binding (association) and unbinding (dissociation) of a targeting agent and a target.

Further disclosed herein is an SDD whereby a change in impedance caused by a binding event between a targeting agent and target is connected electrically to comparator and latch circuitry that facilitates the detection of the binding (association) and unbinding (dissociation) of a targeting agent and a target.

Also disclosed is an SDD, whereby triggering of latch circuitry causes the signaling circuitry to transmit information regarding one or more binding events to a receiver or transceiver, including over a period of time.

Moreover, the inventors disclose an SDD where the information transmitted by the signaling circuitry of the device regarding binding events may be used to calculate binding affinity and other binding affinity-associated parameters between one or more targeting agents and one or more targets.

Further still, the inventors discloses an SDD where the information transmitted by the signaling circuitry of the device regarding binding events may be used to calculate clinically relevant information regarding the pharmacokinetics of the targeting agent, the target, or both, including the equilibrium dissociation constant, K_{D}.

The inventors also disclose an SDD where the information transmitted by the signaling circuitry of the device regarding binding events may be used to calculate biologically or clinically relevant information regarding the binding kinetics/affinity of the targeting agent, the target, or both, or association (proportional to Kₒₙ) and dissociation (proportional to K_{off}) and occupation time (i.e., residence time, τ).

The inventors further disclose an SDD where the information transmitted by the signaling circuitry of the device regarding binding events is over a period of time, such that information regarding binding kinetics, such as binding affinity, K_{D}, Kₒₙ, K_{off}, and occupation time, is updated or revised and dynamically calculated over a period of time.

Moreover, the inventors disclose an SDD where the information transmitted by the signaling circuitry of the device regarding binding events is over a period of time using a device-associated clock signal, such that information regarding binding kinetics/affinity, such as binding affinity, K_{D}, Kₒₙ, K_{off}, and occupation (or residence) time, is updated or revised and dynamically calculated over a period of time.

Also disclosed herein is an SDD where the information transmitted by the signaling circuitry of the device regarding binding events and associated binding kinetics data (e.g., binding affinity, K_{D}, Kₒₙ, K_{off}, and occupation time) is output, optionally further processed, and provided to a user on a computing or display device.

The inventors also disclose an SDD where the information transmitted by the signaling circuitry of the device regarding binding events or associated spatial genomics data (e.g., protein folding geometry or configuration) is output or displayed to a user, including without limitation on a laptop computer, desktop computer, video monitor, tablet, or smartphone device.

Further disclosed herein is an SDD that further includes a unique identifier or code that can be read or detected by a receiver to distinguish one SDD from another SDD.

The inventors also disclose an SDD that is powered by power circuitry. This power circuitry may power the SDD (1) inductively (e.g., from a tuned RF source) through a power harvesting coil integrated into the device, (2) by a DC power source, such as an integrated battery or through a tethered power supply cable, and/or (3) by an AC power source.

Moreover, the inventors also disclose an SDD that may include signaling circuitry to receive input signals (for example, control signals from an associated computing or display device) and transmit output signals (for example, binding event-related data to an associated computing or display device).

Still further, the inventors disclose an SDD that may include signaling circuity that may further include a modulator for encoding and/or transmitting data.

Moreover, disclosed herein is an SDD that may include pad addressing circuitry.

Still further, disclosed herein is an SDD that may include an effecting subsystem, further including electroporation or electroablation needles capable of electroshocking or electroporating a bound cell.

The inventors also disclose an SDD that electroporates or electroablates a target cell using an effecting subsystem, allowing device access to cell contents, including mRNA.

The inventors further disclose an SDD with poly-thymine capture sequences on the device surface that bind to a cell's mRNA accessed via the device's effecting subsystem.

Also disclosed herein is an SDD that can be collected after use in living tissue, allowing mRNA bound to the device to be retrieved and sequenced using RNA sequencing techniques.

Further still, the inventors disclose an SDD that can be tracked in a three-dimensional space, including by using near-infrared fluorophores, computed tomography of RF signals from the device(s), or antenna-based spatial localization techniques.

These and other features and advantages that may be included as part of the invention are described in greater detail below.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings. In accordance with common practice, the various illustrated features in the drawings are not to scale but are drawn to emphasize specific features relevant to the invention. Moreover, the sizes of features and the thicknesses of layers may depart substantially from the scale with which these are shown.

FIG. 1 illustrates an exemplary architecture and layout of the sensing, deciding, and optional effecting subsystems of an SDD.

FIG. 2 illustrates an exemplary architecture and layout of the sensing and deciding subsystems of an SDD without the optional effecting subsystem.

FIG. 3 illustrates an exemplary architecture and layout of an SDD sensing subsystem.

FIGS. 4A-4B illustrate an exemplary embodiment of a sense amplifier circuit with an associated cross-coupled latch of an SDD.

FIG. 5 illustrates an exemplary device with a housing or packaging configured to house an SDD.

FIGS. 6A-6B illustrate examples of how binding data can be output by a binding event sensing device such as an SDD.

FIG. 7 illustrates an example data format for binding measurement data that can be output by a binding sensing device such as an SDD.

FIG. 8 illustrates an example of how a subset of a data packet or binary file may be converted or processed by a receiving computing device and output to a user.

FIG. 9 illustrates another example of how a data packet or binary file content may alternatively or additionally be converted or processed and output to a user.

FIG. 10 illustrates an example process flow for a method of using a binding event sensor device.

FIG. 11 illustrates another example process flow for a method of using a binding event sensor device.

FIGS. 12A-12D illustrate examples of signal spatial localization techniques that may be employed with some embodiments of the present invention.

FIGS. 12E illustrates an example of how bidirectional communications can be carried out to interrogate an SDD.

FIGS. 12F-12G illustrate examples where concentric antenna arrays are used with SDDs.

FIG. 12H illustrates another example of a spatial localization mode that can be used to spatially localize SDDs.

FIG. 13 illustrates an example of using an RF energy harvesting circuit to support bidirectional communications between a binding event sensor device and external system(s).

### DETAILED DESCRIPTION OF EXAMPLE EMBODIMENTS

Described herein are bioanalytic, diagnostic, and/or therapeutic devices, and methods of manufacture and use thereof, that interact with, sense, and identify chemical and biological materials and can detect and report data indicative of the occurrence, magnitude, and duration of one or more binding events between a targeting agent and a target. The system may use binding event-related data further to generate chemically, pharmacologically, and/or clinically relevant information regarding the binding affinity (“K_{D}”) of the targeting agent and the target. As used herein, the term “binding event” may refer to either the binding or association, or the unbinding or dissociation, of a targeting agent and a target.

Binding event-related data may be used further by the system to generate biologically and/or clinically relevant information regarding spatial-temporal multiomics within a cell, tissue, or organ, including linking phenotype with the genotype in a spatially and temporally resolved manner. Bound biological materials can include materials from inside a target cell, tissue, or organ, which may be accessed via electroporation or electroablation by the device. Bound biological materials can be recovered and further analyzed (e.g., by RNA sequencing, mass spectroscopy, binding affinity assays, etc.) to provide information about the cell, tissue, or organ, such as gene transcription (mRNA content) and protein expression. The invention may gather genotypic and phenotypic information over multiple samplings without irreversibly damaging the cell, tissue, or organ, thus allowing for temporally resolved spatial genomic qualities within the target cell, tissue, or organ. Thus, the invention relates generally to determining the genotype and phenotype of living systems (cells, tissues, or organs), including information about a gene and gene product's (i.e., protein) specific location over time.

The invention may be utilized in an in vitro or in vivo environment, including with live cells, animals, and humans. In one embodiment, the invention may be utilized or incorporated in a small and portable form-factor device, with on-chip and off-chip components, for the purpose of drug discovery and validation, reducing the high cost and time duration associated with screening and validating drug candidates. As is known in the art, the process of target validation, compound screening, and lead optimization, with presently utilized systems and methods, can require screening thousands of candidates and many years of effort, and the present invention may be utilized to reduce this costly and time-consuming effort.

Various embodiments of the present invention provide a system, apparatus, and method that can interact with, sense, and identify chemical and biological materials and detect the occurrence, magnitude, and duration of one or more binding events between one or more targeting agents and one or more targets. In some embodiments, the system, apparatus, and method may signal or communicate data, either by wire (e.g., cable) or wirelessly, regarding one or more binding events to an external receiver or transceiver for further analysis, processing and presentation. Binding event data may be used further by the system to generate biologically, pharmacologically, or clinically relevant information regarding the binding affinity, K_{D}, of the targeting agent and the target and information regarding spatial-temporal multiomics of the target, including linking phenotype with the genotype of a target in a spatially and temporally resolved manner. The biological materials bound to the system may be recovered for further analysis, such as sequencing in the case of DNA and RNA or further mass spectroscopy, NMR, or other binding affinity assays in the case of proteins. Thus, the invention relates generally to determining the detection of STM qualities of a target. The invention may be utilized in an in vitro or in vivo environment.

### Definitions

The following terms and phrases have the meanings indicated below unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa unless expressly indicated to the contrary.

As used herein, the singular forms “a,” “an,” and “the” include plural referents unless the context clearly dictates otherwise. For example, reference to “a substituent” encompasses a single substituent as well as two or more substituents and the like.

As used herein, the term “about”, when used in reference to numerical ranges, cutoffs, or specific values, is used to indicate that the recited values may vary by up to as much as 10% from the listed value. As many of the numerical values used herein are experimentally determined, it should be understood by those skilled in the art that such determinations can, and oftentimes, will vary among different experiments. The values used herein should not be considered unduly limiting by virtue of this inherent variation. The term “about” is used to encompass variations of this sort up to, or equaling, 10%.

The term “attach,” “attached,” or “attachment,” as used herein, refers to connecting or uniting by a chemical bond, link, or force in order to keep two or more components together.

As used herein, “for example,” “for instance,” “such as,” or “including” are meant to introduce examples that further clarify a more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “polymer” indicates a large molecule comprised of repeating structural units (“-mers”) typically connected by covalent chemical bonds.

While this invention is susceptible to embodiments in many different forms, there are shown in the drawings, which will herein be described in detail, several specific embodiments with the understanding that the present disclosure is to be considered as an exemplification of the principals of the invention and is not intended to limit the invention to the illustrated embodiments.

### ABBREVIATIONS

BLM—bilipid membrane.
CK—clock signal.
CMOS—complementary metal-oxide-semiconductor.
DNA—deoxyribonucleic acid.
EEBI—environmental-electronic-binding-interface.
EEBITA—EEBI targeting agent(s).
EEBIMTP—EEBI molecular trim pair.
EEBIL—EEBI linkers.
EEBIP—EEBI pad.
IC—integrated circuitry.
MNSDED—micro/nano-sensing-deciding-effecting-device.
nm—nanometer.
PAC—pad addressing circuitry.
RF—radiofrequency.
RFEHC—radiofrequency energy harvesting circuit.
RNA—ribonucleic acid.
SAR—structure-activity relationship.
SDD—sensing deciding device.
STM—spatial-temporal multiomics.
STR—structure toxicity relationship.

### Overview

Examples of binding event sensors such as SDDs are described herein. For example, a binding event sensor may be based in part on a device or apparatus such as the micro/nano-sensing-deciding-effecting-device (“MNSDED”) disclosed in U.S. Pat. No. 11,879,892, for example. The entire disclosure of U.S. Pat. No. 11,879,892 is hereby incorporated herein by reference. The system may be a device or apparatus using chiral molecular wires to transport electrical charge or an electrical signal disclosed in U.S. Pat. No. 11,674,957. The entire disclosure of U.S. Pat. No. 11,674,957 also is hereby incorporated herein by reference.

The binding detection modality described in the '892 and '957 patents enables extraordinary measurement precision and data quality without the imprecision associated with contemporary binding assay methods. Furthermore, the system can be produced using existing high-volume semiconductor manufacturing methods (as set forth more fully in the disclosure of U.S. Pat. No. 11,674,957), leading to a highly cost-competitive solution compared to current market offerings.

In one embodiment, the system includes a device or apparatus that measures the occurrence of a binding event between a targeting agent and a target based on a sensed change of impedance by the device or apparatus. The sensed change of impedance can be manifested as a voltage signal produced by the binding event sensor. The data indicating the occurrence of a binding event may be transmitted, either by wire or wirelessly, to a receiver or transceiver coupled to a computing device. Additional binding event-related data, for example, clock data associated with binding (or unbinding) events, may also by transmitted. Based on the received binding event data, binding rate constants (kₒₙ and k_{off}), residence time, and binding affinity (K_{D}) of the targeting agent to the target may be calculated and presented (e.g., displayed) in real- or near-real-time (including presentation of calculated values, such as K_{D}, for one or more subpopulations of a heterogeneous sample) to a user. Thus, the invention detects and measures the existence and duration of the electrical signal generated by one or more targeting agent-target binding event(s) (e.g., antigen-antibody binding event). In some embodiments, in addition to measuring the occurrence of a binding event, the system further measures the electrical parameters associated with the event, such as an impedance.

During the drug discovery and lead optimization process, the data generated by the system regarding the selectivity of a molecule to its respective target and the specificity of a molecule to a therapeutic effect may provide critical information necessary to fully optimize Structure Activity Relationship (“SAR”) and Structure Toxicity Relationship (“STR”) of a resulting drug candidate, thus improving drug efficacy and safety in clinical trials and increasing the probability of successful regulatory marketing authorization. For example, and without limitation, the binding affinity-related data generated by the system may reveal the existence of one or more subpopulations of a heterogeneous sample, indicating certain one or more subpopulations are associated with toxic effects, one or more subpopulations are more “active” (have higher binding affinity) and one or more subpopulations are less “active” or inactive (have lower binding affinity).

The SDD, with or without the effecting subsystem, as described herein, may be used in “in vivo” environments, including with live cells. In such use cases, the SDD should be micron to nanometer-scale in size, to meaningfully interact with its biological environment. Such devices may be fabricated using the etch liftoff techniques that are described more fully in U.S. Pat. No. 11,879,892. In one embodiment, the SDD may have a major axis length typically in the range from about 100 nm to about 500 μm. Where electroporation or electroablation of a biological entity (e.g., a cell) is required or desirable, the SDD may have an effecting subsystem, typically comprised of two electrically driven nanoneedles and associated nanoneedle driving circuitry that, when triggered under preset conditions (e.g., the detection of a binding event), will have a voltage applied between the nanoneedles to electroporate or electroablate the associated bound cellular structure.

Alternatively, the SDD, as described herein, may be used in “in vitro” (e.g., extracorporeal) environments, including as a laboratory or research diagnostic. In some embodiments, the SDD may be contained within an external casing, housing, or structure to enable or facilitate handling, preparation, and operation by a user. Because the size limitations and packaging requirements are different (and generally, less restrictive) from the in vivo use case, the SDD may have additional functional components or circuitry that are fabricated on-chip (e.g., to enable or facilitate further packaging), and instead of etch lift-off, individual SDDs are fabricated through die singulation or wafer dicing using techniques that are well-known in the art.

In some embodiments, an SDD includes a non-conductive housing, enclosure, or structure, having a surface that further contains or encloses supporting logic and other integrated circuitry, at least one electrically conductive pad positioned on, proximate to, or within the housing surface and connected to the logic circuitry and to at least one targeting agent capable of interacting with a target, wherein in response to target interaction, the logic circuitry allows signaling circuitry to transmit information to a receiver or transceiver, either by wire or wirelessly, regarding binding between one or more targeting agents and one or more targets. In some use cases of an SDD, voltage from a needle-driving circuit may be applied to at least one needle.

In another embodiment, the SDD subsystems may be incorporated directly onto a metal oxide semiconductor chip that contains supporting logic and other integrated circuitry, at least one electrically conductive pad positioned on the chip surface and connected to the logic circuitry and to at least one targeting agent capable of interacting with a target, wherein in response to target interaction, the logic circuitry allows signaling circuitry to transmit information to a receiver or transceiver (by wire or wirelessly), regarding binding between one or more targeting agents and one or more targets. Optionally, a voltage from a needle-driving circuit may be applied to at least one needle on the surface of the chip, if so configured. This SDD may be contained within an external casing, housing, or structure to enable or facilitate handling, preparation, and operation by a user. This casing may include connection ports for wires that may transfer information to an external computer and wires that enable the electrical powering of the device.

The various K_{D}-associated values output by the system or apparatus, as described herein, may be used to predict a protein's 3D structure by comparing output data against existing libraries, such as AlphaFold. The magnitude of the binding-induced impedance change in the target-binding molecule may be used to help infer the rearrangement of the binding molecule's structure.

Targeting agents, as described and utilized by the present invention herein, may include any biological, chemical, or biochemical substance capable of interacting, engaging, and/or binding with a specific biological, chemical, or biochemical entity of interest (i.e., a target). These targeting agents may include oligonucleotides or proteins, amongst other biomolecules. A more detailed description of the range of potential targeting agents contemplated within the scope of the present invention is set forth in U.S. Pat. No. 11,879,892, which is incorporated by reference herein in its entirety. The selection of targeting agents to combine with the SDD in its various configurations, as described herein, may be based on the known or anticipated range or types of subpopulations within a heterogeneous testing sample or exposure environment. Thus, an SDD with multiple or multiple variations of targeting agents can provide a broad range of biologically or clinically relevant data related to binding affinity and kinetics or affinity and spatial multiomics qualities of a biological or biochemical sample or environment.

In some embodiments, an SDD has at least one electrically conductive pad positioned on, proximate to, or within the housing surface and connected to the logic circuitry and to at least one targeting agent capable of interacting with a target, wherein each electrically conductive pad is individually addressable and associated with a unique identifier, such that binding event-related data associated with a particular electrically conductive pad (and therefore, its associated targeting agent) may be correlated to a particular targeting agent for meaningful output or display to a user.

In some embodiments, an SDD includes one or more targeting agents and signaling circuitry to transmit information to a receiver or transceiver (e.g., through RF signaling) regarding binding between one or more targeting agents and one or more targets.

In some embodiments, an SDD includes one or more targeting agents and signaling circuitry to transmit by wire or wirelessly (e.g., through RF signaling).

In some embodiments, a change in impedance caused by a binding event between a targeting agent and target is converted to an electrical signal using a differential amplifier circuitry in the SDD that facilitates the detection of the binding of a targeting agent to a target.

In some embodiments, a change in impedance caused by a binding event between a targeting agent and target is detected using a differential comparator and latch circuitry that facilitates the detection of the initial binding of a targeting agent to a target.

In some embodiments, latch circuitry triggering causes the signaling circuitry to transmit information regarding one or more binding events to a receiver or transceiver, including over a period of time.

In some embodiments, the information transmitted by the signaling circuitry regarding binding events may be used to calculate binding affinity and other binding affinity-associated parameters between one or more targeting agents and one or more targets.

In some embodiments, the information transmitted by the signaling circuitry regarding binding events may be used to calculate information regarding the binding strength (affinity and/or avidity) of the targeting agent to the target, including the equilibrium dissociation constant, K_{D}, corresponding to the concentration of a ligand at equilibrium when half its binding sites are occupied.

In some embodiments, the information transmitted by the signaling circuitry regarding binding events may be used to calculate biologically or clinically relevant information regarding the binding kinetics of the targeting agent to the target, including the speed of binding or association (proportional to kₒₙ) and dissociation (proportional to k_{off}). In some embodiments, the information transmitted by the signaling circuitry regarding binding events is over a period of time, such that information regarding binding kinetics and thermodynamics, such as binding affinity (K_{D}), rate constants (kₒₙ and k_{off}), occupation (or residence time, τ), and chirping voltage and impedance is updated or revised and dynamically calculated over that period of time.

In some embodiments, the information transmitted by the signaling circuitry regarding binding events is over a period of time using an SDD-associated clock signal, such that information regarding binding kinetics and thermodynamics, such as binding affinity (K_{D}), rate constants (kₒₙ and k_{off}), occupation (or residence) time (τ), and chirping voltage and impedance is updated or revised and dynamically calculated over that period of time.

In some embodiments, the information transmitted by the signaling circuitry regarding binding events and associated binding kinetics/thermodynamics data (e.g., binding affinity, K_{D}, Kₒₙ, K_{off}, t, chirping voltage, and impedance) is output, optionally further processed, and provided to a user on a computing or display device.

In some embodiments, the information transmitted by the signaling circuitry regarding binding events and associated STM data (e.g., protein/RNA conformation and location within a cell, tissue, or organ) is output or displayed to a user, including without limitation on a laptop computer, desktop computer, video monitor, tablet, or smartphone device.

In some embodiments, the SDD includes a unique identifier or code that can be read or detected by a receiver to distinguish one from another.

### SDD Subsystems and Methods of Operation

While this invention is susceptible to embodiments in many different forms, there are several specific embodiments with the understanding that the present disclosure is to be considered as an exemplification of the principles of the invention and is not intended to limit the invention to the illustrated embodiments.

The following will summarize the main subsystems and components of the SDD, the associated circuitry and non-circuitry-related components, the composition of the components of the SDD, and a description of the methods in which these SDD may be synthesized, organized, specialized, administered, and used. Any examples that are described herein are not intended to limit the scope of the system, apparatus, method, or composition, but rather to provide clarification of a broader concept through specific description.

The SDD of the present invention comprises a composite structure that can exhibit microscale-to-nanoscale dimensions. However, it should be understood that when used for in vitro settings, the overall SDD-based system, with the possibility of additional power, signaling, and other circuitry, may be larger and need not as an overall form factor exhibit microscale-to-nanoscale dimensions (although the sensing and deciding subsystems used for detecting binding events should still exhibit microscale-to-nanoscale dimensions). The portion of the SDD contacting an environment external to the SDD is generally identified as the surface of the SDD. More specifically, an SDD is comprised of transistors and associated integrated circuitry (“IC”) contained within a “housing” of dielectric or any non-conductive material.

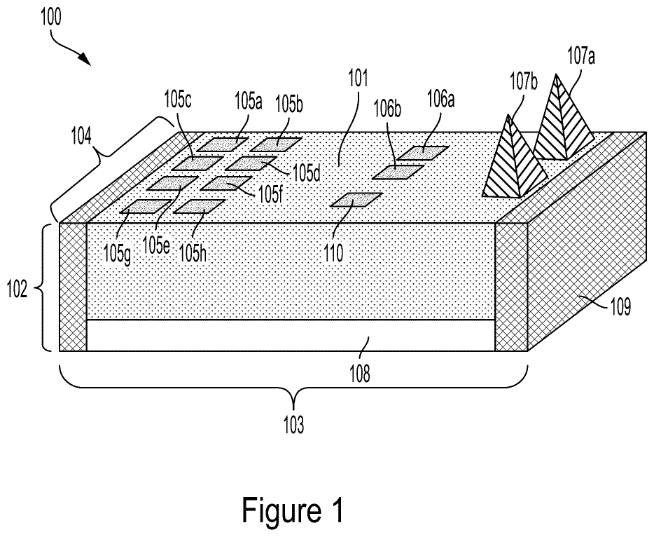

FIG. 1 illustrates the exemplary geometry and surface features of an SDD 100. In this embodiment, the SDD has a spheroidal or cuboidal geometry associated with the etch-liftoff process (for example, for in vivo use cases), and SDD surface 101 is associated with the wafer surface in which the SDD was fabricated. SDD surface 101 is substantially flat. In general, the SDD may be characterized by a SDD major axis length 103 and a SDD minor axis length 104 and has a SDD thickness 102 (z-height). SDD surface 101 contains a plurality of EEBI pads 105a-h, which are electrically conductive areas; a plurality of SDD communication bus pads 106a-b, which are electrically conductive areas that allow the SDD to communicate electrically to a test process; and a plurality of nanoneedle pairs 107a-b, comprised of nanoneedles 107a and 107b, which project up substantially from SDD surface 101. SDD bottom 108 of the SDD is comprised of a material that is not susceptible to the action of an etch liftoff chemistry, and SDD sides 109 may be made of a different material that is also resistant to the liftoff chemistry and provide a substantially hermetic seal for the sides of the device. (If the SDD is being fabricated through die singulation or wafer dicing instead of etch liftoff, sides 109 may be omitted). The SDD top surface 101 also contains a solution-to-bulk silicon connection pad 110, which serves as a potential “ground” reference for electrical measurements with respect to cell surfaces or other surfaces or objects in the biological solution with the SDD. Solution-to-bulk silicon connection pad 110 may be coated with a conductive polymer or a polyelectrolyte layer to modify its electrical properties in solution. Solution-to-bulk silicon connection pad 110 may be electrically connected to the bulk silicon of the SDD.

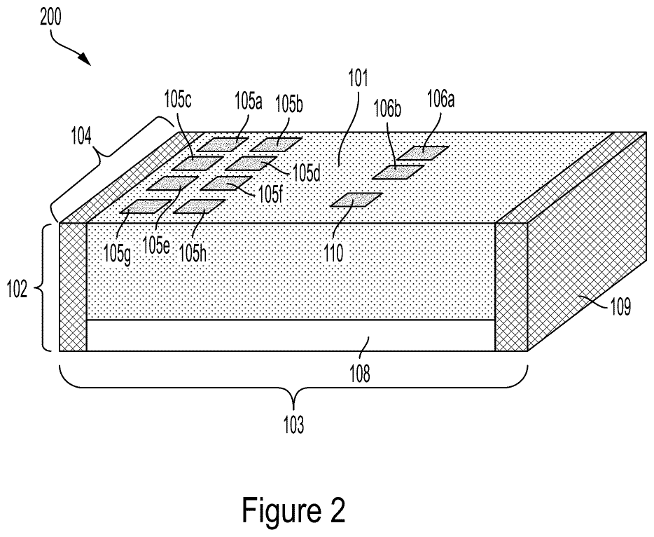

FIG. 2 illustrates an exemplary architecture and a layout of an SDD 200, including exemplary geometry and surface features of an SDD. Like the SDD 100 of FIG. 1, SDD surface 101 is substantially flat and contains a plurality of EEBI pads 105a-h, which are electrically conductive areas. Notably missing from the SDD 200, in contrast to the alternative embodiment SDD 100, are the nanoneedle pairs 107a-b of FIG. 1.

A typical SDD may be divided into a number of subsystems, which interoperate with each other to achieve a desired diagnostic outcome. Each of the subsystems described below may be modified with specific design features to achieve a desired end-use case or outcome. In general, the SDD may be comprised of at least the following subsystems: (1) the sensing subsystem, (2) the deciding subsystem, and (3) an associated power supply and signaling subsystem. The SDD may be comprised further of an effecting subsystem.

### Sensing Subsystem

An SDD of the present invention includes a sensing subsystem. The sensing subsystem may be coated or associated with one or more targeting agents (such as targeting ligands). The one or more targeting agents may include, but are not limited to, antibodies. The SDD sensing subsystem may include targeting agents and electrically conductive linkers that enable sensing of a chemical or biological environment or sample. The SDD sensing subsystem may further include electrically conductive surface components that enable the intake and conversion of the information sensed from the environment or sample into an electrical signal, circuitry that enables or processes logical decisions as to an intended action based on the sensed environment.

Environmental-electronic-binding-interface (“EEBI”) is a term that will be employed herein to describe the assembly of components and materials of the SDD that are associating with, interacting with, and sensing the surrounding environment or sample and, in some embodiments, transferring chemical or biochemical information from this environment or sample to other components of the SDD by converting this chemical or biochemical information into an electrical signal that the SDD electronic components may process. The term “sensing” refers to the specific process of the SDD associating with the environment or sample through specific binding interactions and converting chemical and biological information into electrical information that is propagated into the SDD circuitry.

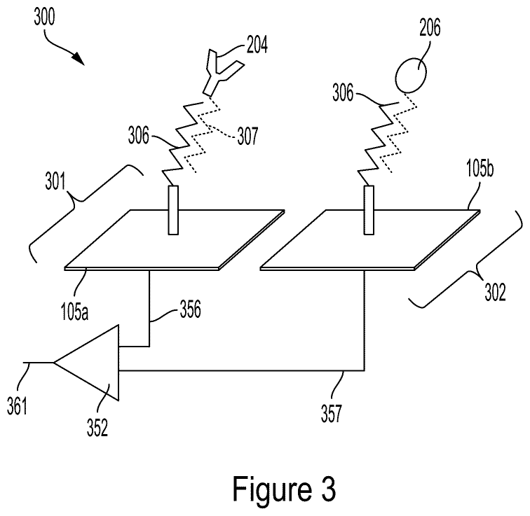

FIG. 3 illustrates a general exemplary architecture and layout of an EEBI molecular trim pair (“EEBIMTP”) 300 for use in the sensing subsystem. The components include: a targeting EEBI 301 that includes a target EEBIP 105 a; a reference EEBI 302 that includes a reference EEBIP 105b; sense amplifier circuitry 352 that has a first input target voltage 356 from the target EEBIP 105 a and a second input reference voltage 357 from the reference EEBIP 105b. The sense amplifier circuitry 352 has an output voltage 361. As shown by FIG. 3, an EEBITA 204 can be associated with EEBIP 105a, and a reference agent 206 can be associated with EEBIP 105b. The output voltage 361 can be a signal that is representative of a binding event between the EEBITA 204 and a sample.

In operation, the EEBIMTP 300 transfers chemical or biochemical information from the environment to other components of the sensing subsystem by converting chemical or biochemical information into an electrical signal that circuitry for the sensing and deciding subsystems can process as described in greater detail in the above-referenced and incorporated '892 patent.

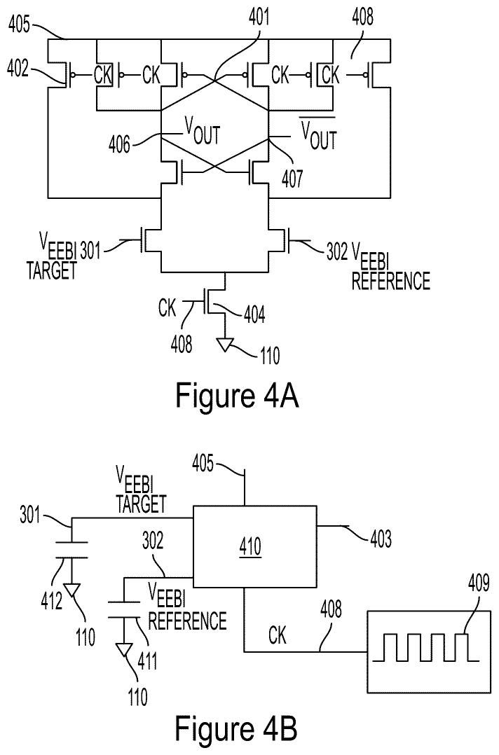

More specifically, EEBI pads 301, 302 are located on the surface of the SDD and comprised of materials that are electrically conductive and whose surface may be chemically modified (as described herein). These materials may include but are not limited to metals (for example, gold, silver, copper, titanium, or other metallic materials known to those skilled in the art of microelectronics manufacturing) and semiconductive materials (e.g. silicon) that are doped sufficiently to become a conductive material, through methods known by those skilled in the art. These EEBIPs will be the direct physical and electrical connection on the surface of the SDD to the SDD integrated circuitry that will be described in further detail herein and in the above-referenced and incorporated '892 patent. In some embodiments, each EEBI pad has a unique identifier and is individually addressable and associated with a unique identifier, and pairs of pads each have a targeting agent and reference agent, respectively, attached as shown by FIG. 3. EEBI pads are electrically connected to amplifier circuitry (e.g., see FIGS. 4A and 4B). Additional background details of EEBI pads, as contemplated and utilized by the present invention, are disclosed in the above-referenced and incorporated '892 patent.

EEBITA 302 is one or more targeting agents (such as targeting ligands), including targeting agents calibrated (selected) to identify subpopulations in a heterogeneous population sample, coupled to a target-associated EEBI pad 301a (“target EEBIP”). Additional background details of EEBI targeting agents, as contemplated and utilized by the present invention, are disclosed in the above-referenced and incorporated '892 patent.

EEBI Molecular Trim Pair (“EEBIMTP) is the term that will be employed to describe the whole assembly of a reference agent 206 (described herein) coupled to a reference-associated EEBI pad 302 (“reference EEBIP”) through the same linker and functional handle-assembly (same EEBIL assembly) that is used to couple an EEBI targeting agent to a target-associated EEBI pad, the voltage of the reference EEBIP being compared to the voltage of a target EEBIP through a differential amplifier (e.g., see 352 in FIG. 3). The EEBIMTP may serve as part of a voltage and/or impedance comparator in the case that a non-specific binding event occurs on the target EEBIP, the voltage and capacitance difference being determined through a differential amplifier as is described further herein. These EEBIMTPs may ensure that, upon non-specific binding of the target EEBIP, there will be a negligible voltage difference (enabled through voltage comparison by the differential amplifier) between the target EEBIP and the corresponding reference EEBIP. This EEBIMTP may, therefore, ensure that the latch (described herein) will not trigger non-specific interaction events associated with the target EEBIP, e.g., a false positive. The EEBIMTP may be calibrated by identifying and binding a “reference” molecule or biomolecule to the reference EEBIP. This “reference” molecule or biomolecule may be tuned or selected so that when it interacts non-specifically with the environment (including non-specific cellular interactions), it will effect a near equivalent voltage change in the reference EEBIP as will be created in the target EEBIP when the EEBITA engages in non-specific interactions. These EEBIMTPs may be configured and designed based on the particular application of the SDD and may be configured and designed through experimental development, with the configuration and design of the EEBIMTP further informed by the particular environment in which the SDD is to be administered. Additional background details of the EEBIMTP, as contemplated and utilized by the present invention, are disclosed in the above-referenced and incorporated '892 patent.

EEBI Linkers (“EEBILs”) may serve to attach a targeting agent to an EEBIP and to carry any electron transfer or charge transfer to the EEBIP arising from a specific binding event. EEBI linkers also may serve to attach a reference agent to an EEBIP in the context of the EEIMTP described herein. The EEBIL may be comprised of any combination of bio-conjugately or chemically linked chiral molecular wires, small molecules, biomolecules, compounds, polymers, moieties, or other entities, and they may be designed to be as small as practicable (e.g., a small oligomer in embodiments that use polymers as EEBILs) in order to minimize the attenuation of the charge transfer across the EEBIL and increase the ability of the EEBIL to elicit a voltage change at the EEBIP through charge transfer facilitated by the EEBIL when there is a specific EEBITA-to-target binding event. As an example, the EEBILs may comprise a single-stranded DNA (ssDNA) 306 as shown by FIG. 3. A second piece of ssDNA, 307, is rigorously complementary to ssDNA 306, and this ssDNA 307 is bound to ssDNA 306 in the finished device. Additional background details of the EEBIL, as contemplated utilized by the present invention, are disclosed in the above-referenced and incorporated '892 patent. Additional background details of the EEBIL chiral molecular wires to transport electrical charge or an electrical signal are disclosed in U.S. Pat. No. 11,674,957. The entire disclosure of U.S. Pat. No. 11,674,957 also is hereby incorporated herein by reference.

Sense amplifier(s) 352 will be incorporated into the integrated circuitry in order to amplify the voltage difference between any two EEBIPs (e.g., with reference to FIG. 3, the voltage difference between a voltage signal 356 from a target EEBIP and a voltage signal 357 from a reference EEBIP; or the voltage difference between two reference EEBIPs). When an EEBITA on an EEBIP is attached to its target, there will be a change in the electric field distribution around the target EEBIP with associated electrical signal conductance through its corresponding EEBIL, which differs from the field distribution around the reference EEBIP and associated electrical signal conductance through the associated EEBIL with its non-specific background voltage. The resulting difference in target-to-reference EEBIP voltages may be detected and amplified from initial voltage differences and converted to a complementary metal-oxide-semiconductor (“CMOS”) logic level for use by the SDD control circuitry.

In some embodiments, the sense amplifier output 361 may be subject to further signal conditioning (e.g., amplification, filtering) and is then latched into a storage circuit. Regenerative sense amplifiers incorporating clocked cross-coupled pairs with precharge, measure, and latch phases may be employed to boost the small voltage differences between the reference and target EEBIP to CMOS logic levels. These clocked sense amplifiers will output multiple measurements based on one or more clock frequencies. If these varieties of sense amplifiers are employed, a threshold number of “positive” measurements per time interval may be counted by a counting/thresholding circuit that triggers downstream logic upon counting a predetermined number of “binding events”, or multiple measurements of the same binding event. The counting circuit may then send a logical signal to the decision circuitry. This data or logical signal constitutes an input to a signal whether and how long an EEBITA has bound to its specific target. Additional background details of the amplifier circuitry, as contemplated and utilized by the present invention, are disclosed in the above-referenced and incorporated '892 patent.

### Deciding Subsystem

The SDD deciding subsystem may be comprised of differential amplifier/comparator/latch and clock circuitry that facilitates the detection and measurement of the magnitude and duration of an EEBITA's binding to a target.

Decision logic circuitry may be employed within the SDD to facilitate the logical processing of combinatoric states, for instance, when a single or group of distinct EEBITAs have bound (or not bound) their respective targets. The term “bar code type recognition” as used herein refers to a logic-based decision made by the SDD integrated circuitry that one or more EEBITAs have bound (or not bound) their respective targets and further initiate an effector mechanism based on a predefined logical combination of triggered (and un-triggered in some cases) latches that are specific to an ensemble of EEBITA binding events and possibly the lack of EEBITA binding events.

The sense amplifier (“SA”) functions as a comparator, which changes the logical state of its output upon detecting a sufficiently large difference between the targeting EEBI and reference EEBI. Changes in capacitance upon binding of a target at the targeting EEBI will induce a voltage difference between the targeting EEBI and the reference EEBI, V_{EEBI target}−V_{EEBI reference}, which is amplified and detected by the SA supplied with a clock signal.

An example of an SA circuit that can be used as part of the decision logic circuitry is illustrated in FIG. 4A. The SA differentially amplifies voltages from a pair of EEBIs, targeting EBBI 301 (V_{EEBI target}) and reference EEBI 302 (V_{EBBI reference}) (corresponding to targeting EEBI 301 and reference EEBI 302 in FIG. 3), and incorporates a cross-coupled latch (401) with output 406. The SA functions as a comparator, which changes the logical state of its output upon detecting a sufficiently large difference between EEBI 301 and EEBI 302. Changes in capacitance upon binding of a targeting agent at the targeting EEBI 301 will induce a voltage difference between the targeting EEBI 301 and the reference EEBI 302, V_{EEBI target}−V_{ERBI reference}, which is amplified and detected by the SA (410—see FIG. 4B) supplied with clock signal 408. Clock signal 408 (node CK) enables NMOS transistor current source 404 and PMOS current sources 402 during linear amplification and regenerative amplification phases. Power is applied at 405 and the output voltage, Vₒᵤₜ, is measured at 406. The complement of Vₒᵤₜ, V_{outbar} (measured at 407), is 180 degrees out of phase with Vₒᵤₜ. A latch circuit or other logic elements will be connected to 406 (Vₒᵤₜ) and 407 depending on the final detection scheme. The output of the latch circuit 406 represents the binding detection signal. A schematic of a detection circuit incorporating a sense amplifier is represented in FIG. 4B by the sense amplifier 410, with representative capacitors 412 and 411 representing capacitance at the targeting EEBI and reference EBBI, respectively. Power is applied at 405, and the SA binding signal is output at 403. Sense amplifier 410 is clocked from clock circuit 409 which outputs clock signal 408. All of the sensing circuitry is referenced to solution-to-bulk silicon connection pad 110.

In some embodiments, if the sense amplifier detects a voltage difference above a predefined or reference threshold, it triggers an integral latch that sends an electronic signal to the SDD logic circuit, indicating that a binding event has occurred. In other words, the output of the latch circuit 406 represents the binding detection signal. This electronic signal may be output to a user (for example, through signaling circuitry, as described herein), indicating that binding of a target associated with kₒₙ has occurred (for example, indicated on an associated display device).

In some embodiments, if the voltage difference between the targeting EEBI and the reference EEBI subsequently falls below a predefined or reference threshold, the integral latch or latch circuit may “reset,” indicating that an unbinding of a target associated with k_{off} has occurred. The associated electronic signal also may be output to a user device (for example, through signaling circuitry, as described herein).

In some embodiments, the sense amplifier is clocked from a clock circuit 408 which outputs a clock signal 409 as noted above. Upon binding of a target molecule to a target EEBI, variations in the capacitances between the target EEBI and associated reference EEBI will lead to different discharge rates, leading to a time-varying voltage difference varying with the clock signal output from the clock circuit, which will then be detected by the sense amplifier. This time-varying voltage difference may be used to calculate the magnitude of a binding event as well as a targeting agent's residence time, i.e., the time the targeting agent spends in contact with its target. This associated electronic signal may also be output, through signaling circuitry, to a user device (for example, through signaling circuitry, as described herein).

Additional background details of the amplifier/comparator/latch and clock circuitry, as contemplated and utilized by the present invention, additional embodiments utilizing two or more dedicated sense amplifiers, are disclosed in the above-referenced and incorporated '892 patent. Variations to and deviations from these deciding subsystem exemplary circuits should be readily apparent to one of ordinary skill.

### Power and Signaling Circuitry

Power to the SDD's subsystems and subsystem components is provided via power circuitry and may be supplied inductively (e.g., from a tuned RF source) to a power harvesting coil integrated into the device. Additional details of how a power harvesting coil may be incorporated into an SDD and subsequently powered by a tuned RF source are disclosed in the above-referenced and incorporated '892 patent. Alternatively, power to the present invention's subsystems and subsystem components may be supplied by a DC power source, such as an integrated battery, or through a tethered power supply cable (e.g., a USB-C cable connecting the invention to a laptop computer). Alternatively, power to the present invention's subsystems and subsystem components may be supplied by an AC power source, although it is acknowledged that an AC power source limits the portability and deployability of the present invention relative to an inductive or portable battery power supply. As should be readily apparent to one of ordinary skill, overall device size, specific use cases (e.g., in vitro or in vivo environments), and specific subsystem configuration power requirements will dictate the size and type of power circuitry and power required to provide functionality to the various SDD subsystems, as contemplated in this present disclosure. In particular, because in vitro use (e.g., a lab bench setting) does not impose the same size restrictions as in vivo use, the SDD system in vitro settings need not be powered by an external, tuned RF source that interacts with an internal harvesting circuit but may instead be powered by an external power source, such as that provided by a battery, or a tethered smartphone or tablet via a USB-based cable.

In some embodiments, an SDD also includes signaling circuitry to receive input signals (for example, control signals from an associated computing or display device) and transmit output signals (for example, binding event-related data to an associated computing or display device). In some embodiments, control signals may include, without limitation, operating parameters that are input by a user, such as the initiation or total duration of a test, sample rate, or selection of a subset of total available EEBIPs. In some embodiments, output signals may include, without limitation, a unique SDD ID (including for authentication/authorization purposes), unique pad ID (sufficient to uniquely identify each pad), clock data, pad binding state (e.g., bound or unbound), binding signal magnitude, and so on.

The signaling circuity may include a modulator for encoding and/or transmitting data that identifies a specific SDD (i.e., a unique device identifier, such as a digital device serial number), as well as individual EEBI pad identifiers, binding event-and binding status-related data (such timing and magnitude of binding events associated with a particular, addressable EEBI pad), clock data, and so on. As contemplated herein, the signaling circuitry may transmit and receive signals with an external receiver or transceiver over a wired or wireless connection (e.g., Bluetooth or Wi-Fi). Wireless connectivity of the present invention may be provided through a wireless chipset and associated antenna for wireless signaling or maybe read through an RF reader, as dictated by the particular invention deployment use case, data throughput requirements, cost, and so on.

In some embodiments, an SDD has one or more pairs of EEBI target pads and associated reference pads (EEBI pad pairs), and the signaling circuitry of the present invention further comprises pad addressing circuitry (“PAC”). The pad addressing circuitry may utilize a matrix addressing scheme or a multiplexer-based addressing system. In one embodiment, the EEBI pad pairs may be arranged in a grid-like pattern with rows and columns. The pad addressing circuitry may apply voltage to a specific row or rows while grounding or sensing at a specific column or columns, allowing for individual EEBI pad(s) addressing and readout. In some embodiments, the pad addressing circuitry may use one or more multiplexers (MUX) to select which EEBI pad (or EEBI pad pair) to interact with, wherein the one or more multiplexers receive binary address input(s) and route a signal to or from a specific EEBI pad. The pad addressing circuity may include clock circuitry, outputting a clock signal that can be used to scan (e.g., sequentially) the EEBI pads in a sequential (step) fashion. The pad addressing circuity may include a shift register or counter to assist with the generation of EEBI pad selection signals, and the selected EEBI pad's voltage (or amplified voltage, or other state-associated voltage) is read and sampled using an analog-to-digital converter (“ADC”). In some embodiments, the pad addressing circuitry may include sample-and-hold circuitry for more stable reading through the use of a Metal Oxide Semiconductor Field Effect Transistor (MOSFET) switch, capacitor(s), and operational amplifier, as should be readily apparent to one of ordinary skill.

Two or more EEBI targeting pads may be addressed serially or in parallel, depending on overall device configuration, using addressing circuitry techniques. In some embodiments, cache controller circuitry, including internal cache (buffer) memory, may temporarily store a plurality of target pad data or readings so that the data may be communicated via signaling circuitry to an external receiver or transceiver. Exemplary use of cache controller circuitry and cache memory for reading and temporarily storing read data values is set forth in U.S. Pat. No. 5,434,990, which is incorporated herein by reference.

It should be readily apparent to one of ordinary skill that specific aspects of the power circuitry may be fabricated “on-chip” with the sensing and deciding subsystems, and other aspects of the power circuitry, may be “off-chip,” as long as the “on-chip” and “off-chip” aspects, in combination, achieve the power circuitry functionality described herein.

Furthermore, it should be readily apparent to one of ordinary skill that specific aspects of the signaling circuitry, including pad addressing circuitry, may be fabricated “on-chip” with the sensing and deciding subsystems, and other aspects of the signaling circuitry, including pad addressing circuitry, may be “off-chip,” as long as the “on-chip” and “off-chip” aspects, in combination, achieve the signaling circuitry functionality described herein.

### Device Housing or Packaging

In some embodiments, and depending on the use case (e.g., in vitro use cases), the assembly of SDD subsystems (e.g. the EEBI and associated circuitry described herein) may be embedded in a chip form factor and housed inside a package (external casing) with deliberately placed intake ports and microfluidic channels to enable the device preparation and the introduction of reference agents, targeting agents, and/or biologic samples of interest. In some embodiments, one or more ports may be dedicated to introducing biological samples of interest into the device by means of, for example, and without limitation, a pipette or cartridge. In some embodiments, select ports may be “exposed” for click-to-combine agent combination by adhesive covers that may be removed for exposure to appropriate corresponding agents.

For in vitro use (e.g., research laboratory settings), the SDD-based system or apparatus may further be housed within a housing approximately the size of a memory stick or pack of gum, containing further integrated circuitry and exposed pads to receive both reference and targeting agents for “click to combine” chemistry processes. Alternatively, or in addition, the system or apparatus may include microfluidic channels for transferring or transporting a solution (e.g., biologic of interest) to the various pads.

In some embodiments, the external casing may also contain additional circuitry, including, without limitation, portions of the signaling circuitry (including pad addressing circuitry) and power circuitry that are “off-chip,” to be connected with portions of the signaling circuitry (including pad addressing circuitry) and power circuitry that are “on-chip,” i.e., fabricated on the same silicon as the SDD subsystems.

In some embodiments, the external casing may also contain additional circuitry, including, without limitation, a data port (e.g., USB-compliant data port), a power supply port, a wireless chipset, and associated antenna circuitry.

In some embodiments, the housing contains either a wired (e.g., USB-based) interface for communicating to and from a computing device, including, without limitation, a desktop computer, laptop computer, tablet, or smartphone.

In some embodiments, the housing contains either a wireless interface (e.g., Bluetooth) for communicating to and from a computing device, including, without limitation, a desktop computer, laptop computer, tablet, or smartphone.

In some embodiments, the housing is made of any rigid, non-reactive material that can be manufactured to provide the mechanical and thermal management properties to house and protect the components described herein, including protection from mechanical shock (e.g., drops) and thermal stresses. It is further advantageous that the housing provides protection against unintended liquid or gas intrusion. In some embodiments, the housing is made of a plastic. In some embodiments, the housing is made of a metal. In some embodiments, the housing is made of a substrate, such as glass.

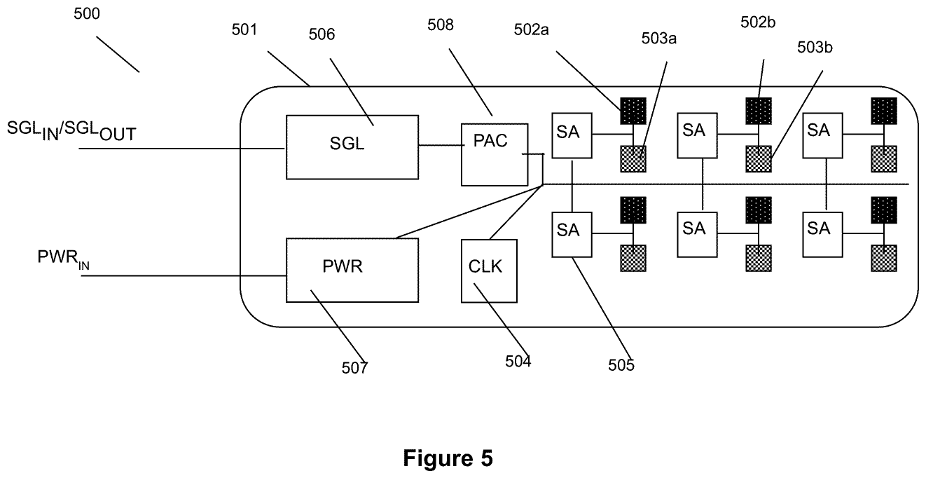

FIG. 5 illustrates an exemplary device with a housing or packaging configured to house an SDD-based system or apparatus of the present invention. In particular, FIG. 5 is a two-dimensional rendering of structural features and functional subsystem blocks of one embodiment of the present invention. For simplicity, not all subsystems, components, and subcomponents as described herein are depicted in FIG. 5. Furthermore, some components or subcomponents of SDD-based device 500 may be fabricated on-chip, or “off-chip”. SDD-based device 500 includes an enclosure or packaging 501 housing one or more EEBI targeting pads 502a, 502b, and so on, that are paired with one or more EEBI reference pads 503a, 503b, and so on. Sense amplifier circuitry 505, coupled to clock circuitry 504, amplifies a differential voltage between an addressed EEBI targeting pad and an associated EEBI reference pad, where these components can be configured and operate as discussed above in connection with like components for FIGS. 1-4B. As shown by FIG. 5, each pair of pads 502a-503a, 502b-503b, etc. can provide their signal outputs to a corresponding sense amplifier (SA) 505, although this need not be the case (for example, a given SA 505 can receive signals from multiple pad pairs 503a, 503b if desired). Pad addressing circuitry (PAC) 508 can be used receive signal outputs from the SAs 505 and multiplex and/or selectively read the signal outputs from the SAs 505. If the voltage difference detected by a given sense amplifier 505 exceeds a predefined or reference threshold (as might occur during a binding event), it can trigger a latch (not specifically shown in FIG. 5; but see FIGS. 4A and 4B as an example), enabling a signal to be output to the user via the signaling circuitry 506 (see SGL_{OUT} in FIG. 5). This signal may be indicative of a bound state between a targeting agent and a target. Should the amplified voltage between an addressed EEBI targeting pad and an associated EEBI reference pad fall below a predefined or reference voltage (as might occur during an “unbinding” event), the latch may “reset”, enabling another signal to be output to the user via the signaling circuitry 506, indicative of an unbound state between a targeting agent and a target.

The signaling circuitry 506 may also be configured to support communications with external system(s) bidirectionally. For example, an incoming signal from an external system (see SGL_{IN} in FIG. 5) can be received by the signaling circuitry 506 and provide commands regarding which of the pads 502 should be read. This signaling circuitry 506 can then interact with pad addressing circuitry (PAC) 508 to enable an addressable “read” of EEBI pad pairs (for example, in a sequential or repeated looped cycle of EEBI pad pairs) and a bound state signal over a data bus. Example configurations for PAC 508 are discussed above.

Power circuitry 507 includes a power supply circuit, which can be a power harvesting system (e.g., RF-induction coil). Alternatively, power circuitry 507 may include or be connected to an external power source (e.g., AC or DC (battery, or computing device tethering cable). Power is distributed throughout the system via a power bus.

Variations to and deviations from this exemplary apparatus (such as the number and configuration of targeting and reference agent pads, signaling circuitry (including pad addressing circuitry), and power circuitry), depending on specific SDD end uses, applications, and requirements, should be readily apparent to practitioners in view of the teachings herein. Such variations and deviations, as would be apparent to one of ordinary skill in view of the present disclosure, are considered to be within the scope of this disclosure.

For example, the system shown in FIG. 5 can provide output signals that are indicative of the occurrence, magnitude, and duration of binding events in any of multiple ways.

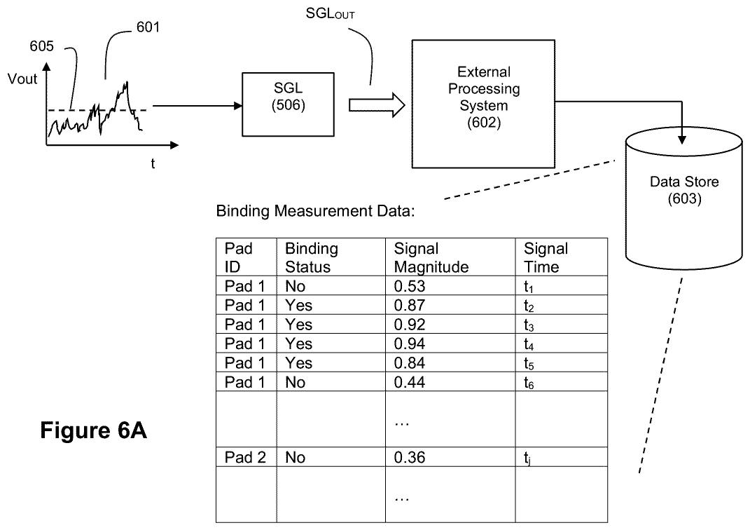

For example, FIG. 6A shows an example whereby the SGL_{OUT} comprises an analog signal that represents the amplified voltage signal over time (see 601) that is indicative of whether binding events have occurred. The analog SGL_{OUT} signal can be received by an external processing system 602 via a wired or wireless interface with the signaling circuitry 506. The external processing system 602 may comprise circuitry (which may include one or more analog-to-digital converters (ADCs) and one or more processors such as CPUs, DSPs, GPUs, FPGAS, and/or ASICs, etc.) that operate to translate SGL_{OUT} into binding measurement data for storage in a data store 603. For example, binding status data that indicates the occurrence of a binding event can be extracted from the output signal based on whether the output signal passes a defined threshold (see 605). The binding status data can be a Boolean value (e.g., yes/no, 1/0, etc.) that indicates whether a binding event has been detected. Signal magnitude data that can represent the magnitude of a binding event can be extracted from the output signal via ADC. Furthermore, signal time data can be associated with each instance of binding status data and signal magnitude data based on a correlated clock signal or the like. The associated signal times for these data values can then be used to determine the duration of any detected binding events.

While FIG. 6A shows an example where SGL_{OUT} is an analog signal representation of 601, and it should be understood that SGL_{OUT} could also be a digital signal representation of 601. For example, the signaling circuitry 506 could include one or more ADCs that operate to digitize signal 601 prior to output as SGL_{OUT}.

FIG. 6B shows yet another example where the device 500 includes a measurement data subsystem 620 that is configured to generate the binding measurement data 626 on the device 500 so that SGL_{OUT} includes a time-series of binding status data values, signal magnitude values, and signal time values. In the example of FIG. 6B, the measurement data subsystem 620 includes an ADC 621 that operates to digitize the signal 601 shown by FIG. 6A and produce a time-series of signal magnitude data values 623. The measurement data subsystem 620 can also (or alternatively) include binding detection logic 622 that uses the techniques discussed above to compare signal 601 with a threshold value (e.g., see 605 in FIG. 6A) to determine whether to assert the binding occurrence data signal 624 to reflect a positive or negative binding status. Further still, a clock signal from clock 504 can be used to correlate the data values with time values. Measurement processing logic 625 can operate to packetize or otherwise generate binary files that hold the clocked magnitude data 623, or binding occurrence data 624, or both. The measurement data subsystem 620 can be embodied by circuitry and logical components on device 500. Furthermore, a cache 627 can be resident on device 500 to hold the binding measurement data 626 while it awaits output as SGL_{OUT} by the signaling circuitry 506.

Data packets or binary files may be comprised of both control information and targeting pad-associated data. Utilizing the processing resources at the receiving computing device (e.g., an external processing system 602 with one or more processors which may comprise compute resource(s) such as CPU, GPU, DSP, ASIC, and/or FPGA resources), data packets (e.g., payloads) or binary files received may be further processed and output in text, graphical, or mixed text/graphical formats at an output device (such as a display) for a user. Alternatively, the data packets or binary files may be subject to further computation, processing, analytics and analysis, including machine learning-based analysis, to output biologically or clinically relevant results to a user on an output device. Such further computation may occur locally (if sufficient local computing resources exist) or in a remote, cloud-based environment.

A simple, exemplary data packet or binary file may be formatted as follows, including both header, metadata, payload, and footer portions, including without limitation the following contents:
SDD ID (including for authentication/authorization purposes)
Pad ID (fixed bit length sufficient to uniquely identify each pad)
Clock Data
Pad Bound State (e.g., 0=Unbound, 1=Bound based on amplified targeting pad voltage exceeding or not exceeding a preset latch threshold, see 624)
Binding Signal Magnitude (see 623)
Packet Length/Footer

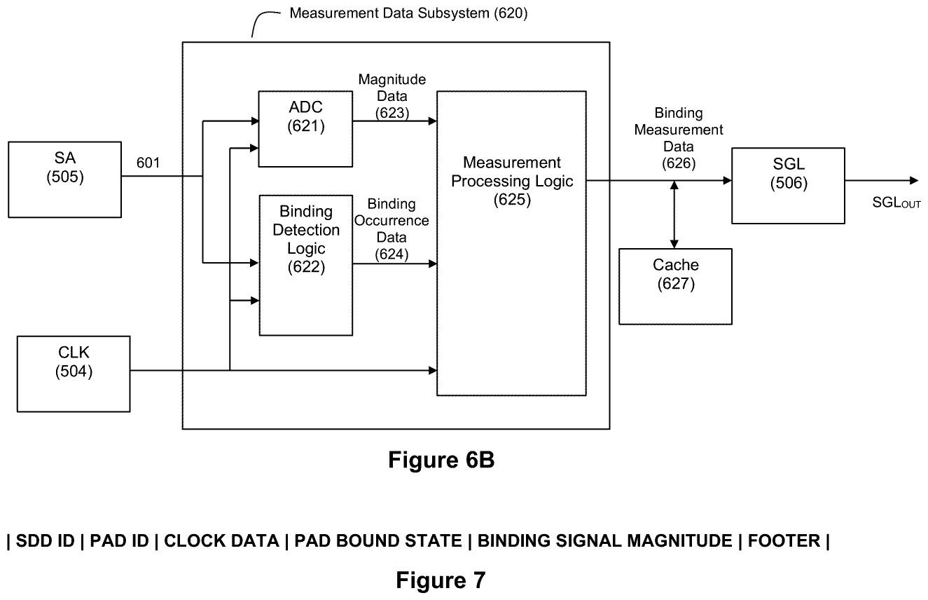

An exemplary conceptual format of the foregoing data packet portions can be as shown by FIG. 7.

The foregoing is merely exemplary, and alternative data formats or binary files for transmitting SDD-derived binding data to a receiver or transceiver for subsequent processing are possible can be employed by a practitioner while falling within the scope of the present disclosure.

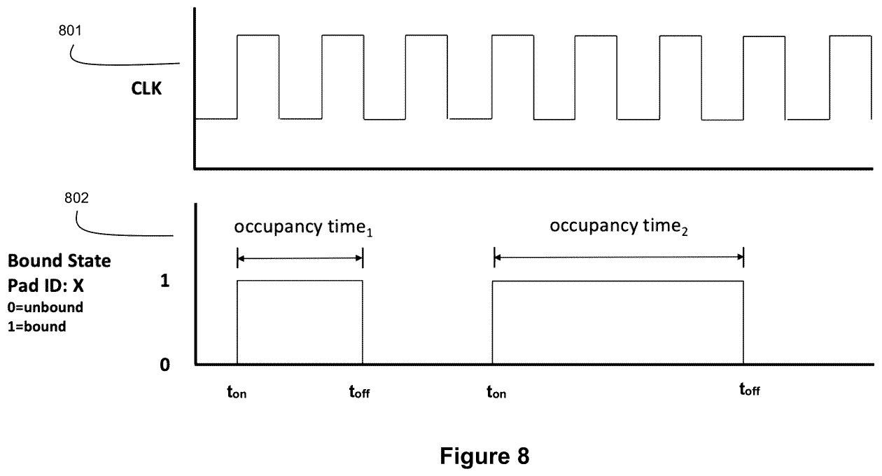

FIG. 8 illustrates how a subset of data packet or binary file content collected by some embodiments of the present invention, including clock data that is correlated to the binding and unbinding of a target (tₒₙ, t_{off}) and occupancy time (τ=t_{off}−tₒₙ), may be converted or processed by a receiving computing device, and output to a user.

With reference to FIG. 8, a connected computing device may optionally display a clock signal in the graphical format 801 depicting the frequency that a given EEBI pad is “read.” In addition (or exclusively), a connected device may process individual pad bound state, for one or more addressable EEBI targeting pads, with correlated clock information to display bound and unbound state(s) and calculated occupancy time(s), on a pad by pad basis 802 (“Pad ID: X,” where “X” is a unique pad identifier). In other words, a user device receiving data from the present invention may calculate occupancy time or times (over a period of time) for a given targeting agent and target (as correlated by specific Pad ID).

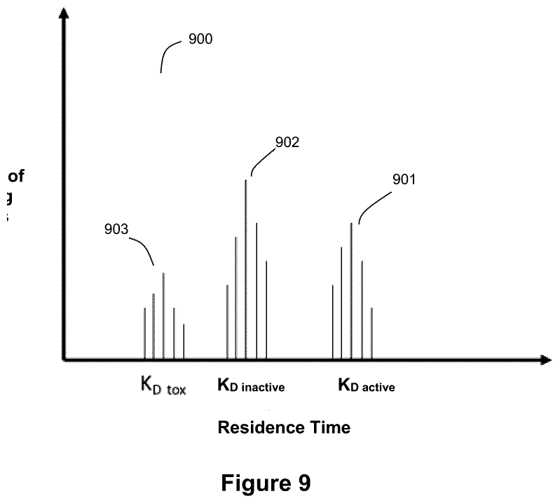

FIG. 9 illustrates another embodiment of how a data packet or binary file content collected by some embodiments of the present invention may alternatively or additionally be converted or processed and output to a user. In the particular embodiment of FIG. 9, the graphical output 900 of binding magnitudes (as represented by impedance or capacitance change due to a binding event) versus associated binding occupancy times, may reveal groupings of sub-populations of a heterogenous sample, providing clinically-valuable insights into whether certain subpopulations of that heterogeneous sample are more “active” (see 901), less “active” such as “inactive” (see 902), or even toxic (see 903).

The foregoing examples of processed SDD data output are merely exemplary, and alternative, scientifically/clinically useful data and data presentation formats are possible while falling within the scope of the present disclosure.

### Effecting Subsystem

In some embodiments of an SDD-type device, electroporation or electroablation needles may be coupled with voltage amplifier circuits that will increase the voltage generated through optional radiofrequency energy harvesting circuitry so that the SDD is capable of electroshocking or electroporating a bound cell. The electroshock or electroporation action may be conducted through a series of no less than two nano-needles—one as the anode needle and the second as the cathode needle (or ground). These nano-needles may protrude from the surface of the SDD, and they may be placed geographically on the SDD surface in a location that is as close as practicable to the pads in order to allow for target pads and their associated reference pads to be in as close proximity as practicable to the nano-needles, also in order to ensure that the nano-needles are in as close proximity to the cell bilipid membrane (“BLM”) as practicable while minimizing the potential for the nano-needles to ablate or porate adjacent cells to the SDD. Thus, the needles electroporate and/or electro-ablate with voltage tuned to provide the desired electroporation or electroablation outcome. The needle-driving circuitry may be coupled to a clock signal to provide an electroporation or electroablation effect for the desired time duration. For example, in response to target interaction with the targeting agent, a sense amplifier connected to the first (targeting) pad and the second (reference) pad is triggered, and deciding logic circuitry can promote at least one of ablation or electroporation of the target. Additional background details of the effecting subsystem associated components and circuitry, as contemplated and utilized by the present SDD invention, are disclosed in the above-referenced and incorporated '892 patent.

### Method of Use Example (Binding Affinity Characteristics)

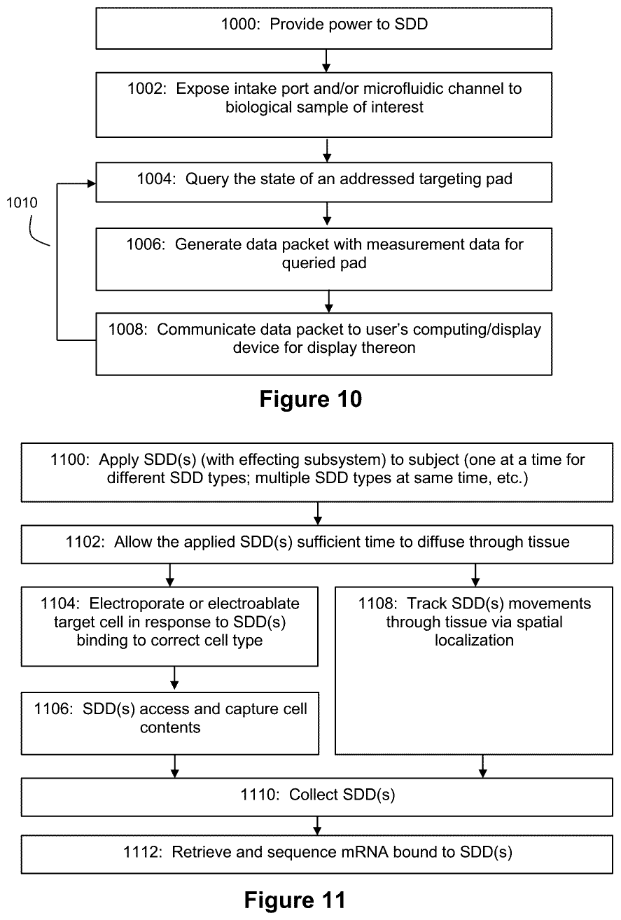

The foregoing example of a specific embodiment is illustrative only and is not intended to be limiting in any way. It is described here to demonstrate a method by which an SDD-based device may be used in a research or drug discovery context, by which the SDD may operate when testing biological or biochemical samples in an in vitro setting. This example as illustrated by FIG. 10 will describe how an SDD (appropriately packaged to enable the functionality as described herein) may be employed in a laboratory or research setting to measure the binding affinity characteristics of a heterogenous biological sample. The following method of use example assumes that, during initial device manufacture and preparation, one or more EEBI targeting pads are provided with one or more targeting agents of interest (corresponding to one or more targets in the heterogenous sample), and one or more EEBI reference pads are provided with reference agents, as more fully set forth in U.S. Pat. No. 11,674,957. Furthermore, as should be understood by one of ordinary skill, certain steps of the following method may be re-ordered; for example, Step 1002 of the following described method may be switched with Step 1000, without materially impacting the overall objective of the invention.

Step 1000: Power is “turned on” or supplied to the SDD-based device, either inductively, via internal battery (DC), or external power source (AC or DC).

Step 1002: One or more intake ports and/or microfluidic channels of the SDD-based device are exposed to one or more biological samples of interest (possibly containing one or more targets). For example, one or more biological samples of interest may be pipetted into one or more intake ports or introduced through a cartridge mechanism coupled to an intake port of the device.

Step 1004: Signaling circuitry, through its pad addressing circuitry, “queries” the bound state of a specifically addressed EEBI targeting pad.

Step 1006: Depending on the latch status of the signaling circuitry, the SDD generates a data packet for output by the signaling circuitry. The data packet includes the targeting pad's bound (or unbound) state, the voltage and impedance (related to ‘chirping’ based conformation detection), and associated clock data correlated to the unique EEBI targeting pad.

Step 1008: One or more data packets is communicated from the SDD and is received at a user's computing or display device, which aggregates, processes, and outputs on the display the measurements and associated calculations (based on the received data packets) either in text, graphical, or mixed format, as may be further selected and formatted by the user.

Step 1010: As controlled by the signaling circuitry, the SDD continues to cycle, “read,” and output data for EEBI pairs (or a subset of EEBI pairs, as may be configured by user or algorithm), per Steps 1004, 1006, and 1008, for a duration of time (as may be configured by user or predefined algorithm). Thus, it should be understood that step 1010 can involve recursive iterations to cycle through reads of different EEPI pairs if desired by a practitioner.

The SDD-based device of the foregoing embodiment may be designed to be used for a single test (i.e., disposable) or may be constructed to be re-used over a plurality of duty cycles after appropriate cleaning and disinfecting.

### Method of Use Example (Spatial Multiomics)

The invention, as described herein, may further be used to collect data pertaining to the spatial expression of the genome, transcriptome, proteome, and post-translational proteome (the ‘multi-ome’). This data can be collected from fixed and living cells in cell culture and tissues and in cells, tissues, and organs in vivo. Further, this data may be temporally resolved in live cells, tissues, and organs both in vitro and in vivo.

SDDs (with nanoneedles as described more fully in U.S. Pat. No. 11,879,892 and hereby incorporated herein by reference) with appropriate surface receptors (i.e., targeting agents) may identify cells of a certain phenotype. Upon identification (i.e., binding) with a cell of a certain intended phenotype, the nanoneedles may be employed to electroporate or electroablate the cell, allowing access to cell contents.

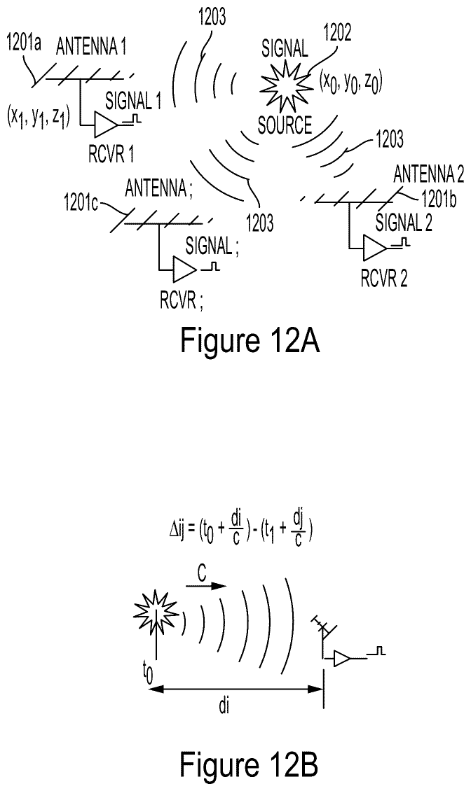

The foregoing example of a specific embodiment is illustrative only and is not intended to be limiting in any way. It is described here in connection with FIG. 11 to demonstrate a method for using an SDD in a research or clinical diagnostic context, by which one or more SDDs may collect data relevant to spatial multiomics.

Step 1100: SDD(s) are applied to a subject. In this example, the SDD(s) can include an effecting subsystem such as nano-needles as discussed above in connection with MNSDEDs. Either one type of SDD can be applied at a time, or several different types (for example with different capture agents to different targets and fluorophores) at the same time, or several different types sequentially in time, and can be applied or used on fixed or live cells, tissues or organs either in vitro or in vivo.

Step 1102: SDD(s) are allowed sufficient time to diffuse or perfuse through the sample (if required).

Steps 1104 and 1106: When the SDD is bound to the correct cell type (identified by the sensing and deciding subsystems), the device may electroporate or electroablate the target cell using the previously described effecting subsystem (see 1104). This will allow the SDD access to cell contents (see 1106), including mRNA, proteins, and other cellular contents. The cell's mRNA will have access to bind to poly-thymine capture sequences on the SDD surface. The cell's proteins may bind to specific capture agents on the SDD surface, including capture agents specific to a mRNA, protein, or post-translational modification of interest. Additionally or subsequently, complementary sequences of specific mRNA transcripts of interest may be coupled to one or more EEBI pads of the SDD, allowing for the detection of mRNA, proteins, or other cellular contents that diffuse through the cell wall pore induced upon electrical stimulation by the SDD nanoneedles.

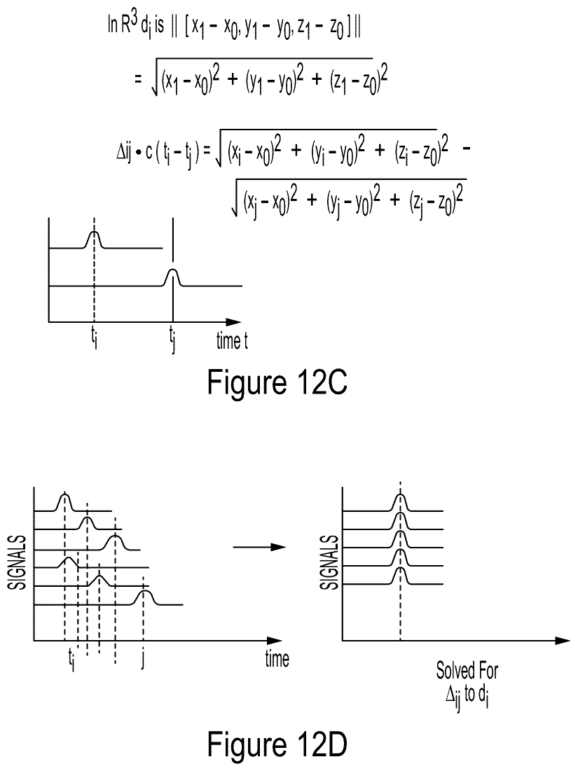

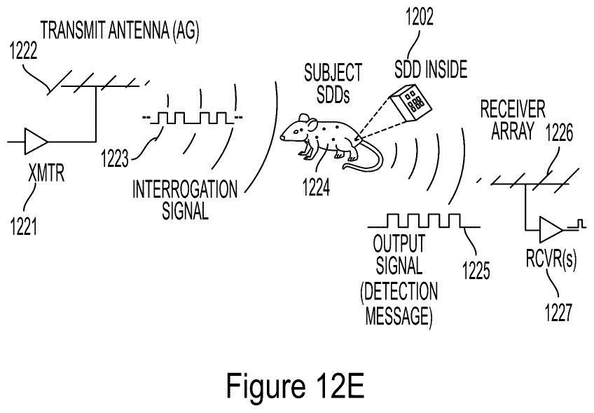

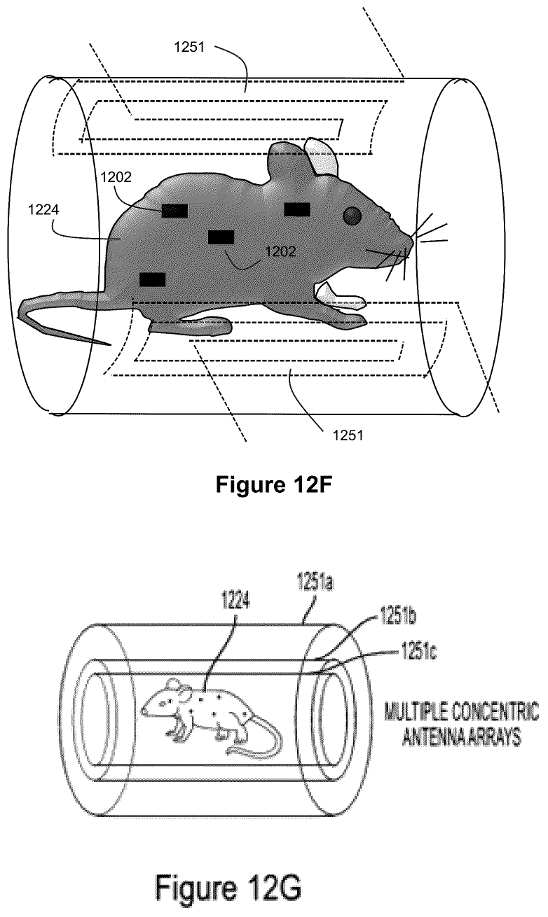

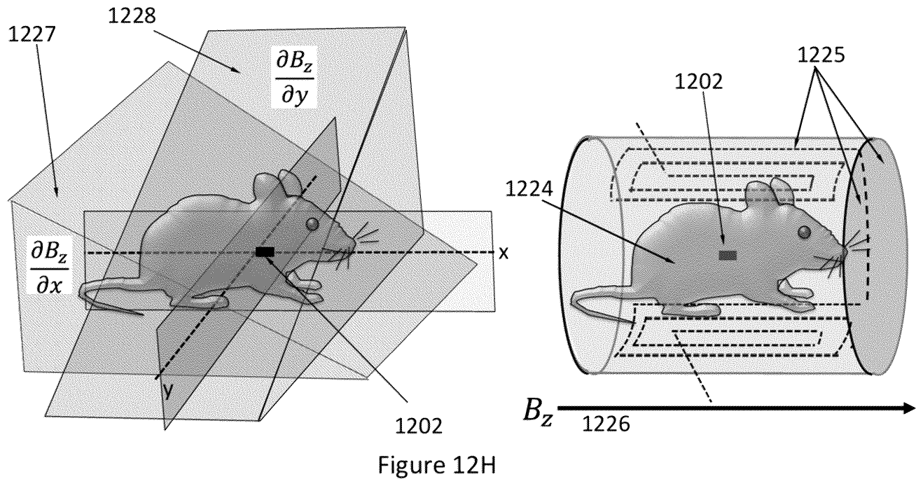

Step 1108: A localization system can be used to track the SDD(s) movements through tissue using spatial localization techniques. For example, if the SDD is labeled with fluorescent tags (for example, with near-infrared fluorophores conjugated to certain capture agents on the SDD), a camera can be used to track the device's movements through tissue through an optical imaging modality, for instance by confocal microscopy. Each SDD may also be localized in 3-dimensional space and over time by placing the sample (cell culture, tissue, organ, or animal/human) in a magnetic resonance imaging system, where each individual SDD position can be identified through both magnetic and RF signaling, for example, the SSD could detect and signal out the magnitude of the magnetic field components at its location, thus establishing its position with respect to MRI gradient fields. FIG. 12H illustrates one such spatial localization mode. The test subject 1224 (with at least one SSD 1202 onboard) is inside a typical MRI static and RF magnetic field assembly 1225, the design details of which will be known by those skilled in MRI equipment design. The main uniform MRI magnetic field Bz (along the axial direction of the MRI assembly) 1226 is modified by gradients in the x 1227 and y 1228 directions, which allow the SSD to localize itself in a single plane in the test subject, as the main field is decreasing predictably along the x and y axes. This localization mode can be extended to three dimensions with another gradient. If desired, the optical image and spatial image from the magnetic and RF signaling apparatus may then be overlayed through software. Each SDD may further be able to transmit an RF signal with information that includes what has been bound (RNA sequences or specific proteins), to which specific EEBI pads on the SDD are bound, as well as the device's unique identifier (as described more fully herein).

Steps 1110 and 1112: SDD(s) may be collected for further analysis (see 1110), for instance, by extracting mRNA bound to poly-thymine sequences on the surface of the SDD(s) and amplifying and sequencing these mRNA using methods such as polymerase chain reaction (PCR) techniques (see 1112). This method of use allows poly-thymine sequences on the surface of the SDD to capture the poly-adenylated tails of mRNA, allowing the full transcriptome of a cell to be collected, sequenced, and cataloged.

This method of use can allow for rapid detection and the ability to transmit, through RF signaling, the presence or absence of a transcript or other cell content of interest. The SDD may further have both the complementary strands for mRNA transcript detection and separate binding pads that have capture agents for the gene product (translated protein) of the mRNA transcript and capture agents for any post-translational modification of interest. This will enable, for the first time, real-time monitoring of the transcriptome and proteome of internal cell contents (both proteins and mRNA) within 3-D matrices, tissues, and organs, including ultimately in humans. SDDs can be located/mapped in situ (and in vivo) through computed tomography of the RF signaling from the SDD. This allows the monitoring of gene transcription and translation and 3-D mapping of the transcriptome/proteome in real-time and in three-dimensional space.

Importantly, selective tuning of the voltage applied to the cell may allow for transient electroporation without the ablation of the cell. This may allow for the monitoring of the internal contents of the cell without irreversibly ablating the cell and further allow for the probing of the cell for future monitoring with a subsequent electroporation event. The system, apparatus, and method of this invention may ultimately enable spatial-temporal studies of gene transcription and gene product expression in vivo.

Single-cell resolution may be accomplished when one SDD carries one cell's multi-omic information.

The system, apparatus, and method of this invention can be used to identify immune cells that interact with tumor cells (tumor-immune interactome). This information is important for developing therapeutics. However, current spatial genomics approaches lose much of this information due to the extensive tissue processing required.

Many different SDDs can be applied simultaneously or sequentially for the purposes of accomplishing the objectives of this system, apparatus, and method.

SDDs, as described herein, can be additionally labeled with NIR (near-infrared) fluorophores to allow tracking as they move through tissue. SDDs can also be spatially monitored through computed tomography of RF signals from the SDDs, wherein each SDD has a unique RF signal identifier, as described more fully below.

### Spatial Localization of Device Signals

According to one embodiment, one or more antenna arrays may be used to localize (locate) the location of an SDD in a three-dimensional space. In such use cases, for example, the SDD may be located inside a biological organism, organ, or tissue. Further, the location of a particular target (mRNA, protein, or post-translational modification) within a cell may be collected by sending through an RF signal from the SDD information on the particular EEBI pad whose targeting agent has been bound by a target, thus providing spatial information relative to the SDD surface. The ability to correlate the location of an SDD, and the expression and location of multi-omic cellular components within the cell itself, in real-or near real-time, in a three-dimensional spatial context is particularly valuable to drug development, scientific research, and clinical diagnoses.

As noted above, a spatial localization system can be used to determine the location of a binding event sensor such as the SDD in three-dimensional space. The spatial localization system can be configured to (1) sense one or more characteristics of the binding event sensor, (2) identify the binding event sensor based on the sensed one or more characteristics, and (3) determine a spatial location in a three-dimensional space for the identified binding event sensor based on the sensed one or more characteristics. The spatial localization system can include one or more processors (e.g., compute resources such as CPUs, DSPs, GPUs, ASICs, FPGAs, etc.) that process signals sensed by the spatial localization system to identify the binding event sensor and determine its location in 3D space.

As an example, the spatial localization system can employ an optical imaging modality (e.g., fluorescence) that detects optically-readable indicia on the SDD(s) (e.g., fluorescent markers) to help determine the SDD(s)'s spatial location in 3D space.

As another example, the spatial localization system can comprise an array of antennas and signal processing circuitry that spatially localizes the binding event sensor based on times of arrivals at the antennas for an RF signal from the binding event sensor. According to one embodiment, as illustrated in FIG. 12A, antenna arrays may be used to determine a signal's bearing using time of arrival. For example, at least three antennae, 1201a, 1201b, and 1201c (and so on), are arrayed in a three-dimensional space sufficiently proximate to the signal source 1202 (e.g., SDD) to receive an output signal 1203 from the SDD. The differences in the arrival time of a signal output 1203 from SDD 1202 at antennae 1201 may be used to locate the SDD 1202 in three-dimensional space based on the time the source device 1202 emits the signal, differences in the arrival time of the signal at the various antennae 1201, and the propagation speed of the medium between the source 1202 and antennae 1201.

As an example, the signal source 1202 may be located by the antenna arrays 1201 from the arrival time (tᵢ) at the i^{th} antenna. Specifically, as shown by FIG. 12B, the differences in arrival time at antennas i and j can be computed from to (which is the time that the source 1202 emits signal 1203), dᵢ (which is the distance from the source 1202 to the i^{th} antenna), dⱼ (which is the distance from the source 1202 to the j^{th} antenna), and c (which is the propagation speed of the signal 1203 through the applicable medium, which may be assumed to be uniform for the path between the source 1202 and antenna 1201). FIG. 12C shows an example of how Δij can be computed for points in an x,y,z coordinate system.

Since the differences between pairs of receivers are used, one Δij becomes the reference signal, and for N receivers, this means that N*((N−1)/2) pairs of Δij are obtained.

The final position can be found by solving a set of equations to extract each dᵢ or by cross-correlation techniques, including modeling the propagation channel between the source(s) 1202 and each receiver in the array and minimizing differences in the cross-correlation (see FIG. 12D).

An SDD equipped with a signaling circuit (e.g., a transceiver) can respond to an RF-based message directed to it from a directional or omnidirectional antenna with a pre-programmed ID code (interrogation message) and detection indication message. For example, FIG. 12E shows an arrangement where the SDD(s) 1202 inside a subject can be interrogated via a transmit antenna 1222 coupled with a transmitter 1201 to communicate an interrogation signal 1223 for reception by SDD(s) 1202. In response to the interrogation signal 1223, the SDD(s) 1202 can transmit an output signal 1225 for reception by receive antenna(s) 1226 and processing via receiver(s) 1227. The device's responding detection message can be located using the foregoing three-dimensional localization techniques discussed herein. Furthermore, an SDD 1202 can be programmed to respond to the interrogation message only if it has detected its pre-programmed target (e.g., protein) or DNA/RNA sequence (e.g., the output signal 1225 can be a detection signal). FIGS. 12F and 12G show examples of antenna arrays 1251 that can be used to communicate with SDD(s) 1202 in this fashion. For example, FIG. 12F shows an antenna array 1251 positioned around a test subject 1224 with multiple SDDs 1202 distributed inside of it. FIG. 12G shows a further example where multiple antenna arrays 1251a, 1251b, and 1251c are positioned around the test subject 1224. These antenna arrays can be concentric with different distances for each antenna to the test subject to support spatial localization as per FIGS. 12A-12D.

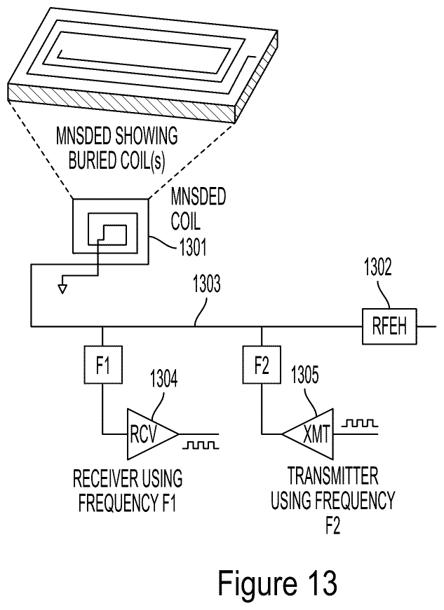

According to some embodiments, and as further shown in FIG. 13, the signaling circuitry of the SDD may leverage power harvesting coil(s) 1301 and an RF energy harvesting circuit (“RFEH”) 1302 of the power circuitry with a diplexer or frequency-specific multiplexer circuits to receive and transmit signals. The RFEH 1302 can be configured as described in the above-referenced and incorporated '892 patent. For signal reception by the SDD, the coil 1301 can be used to receive an RF signal from an external source using a first frequency (F1), where the voltage signal 1303 corresponding to the received RF signal is processed via receiver 1304 using frequency F1. For signal transmission by the SDD, a transmitter 1305 applies a voltage signal 1303 to the coil 1301 using a second frequency (F2). The coil 1301 can then transmit an RF signal using the second frequency F2.

### Summary

While specific embodiments have been illustrated and described above, it is to be understood that the disclosure provided is not limited to the precise configuration, steps, and components disclosed. With the aid of the present disclosure, various modifications, changes, and variations apparent to those of skill in the art may be made in the arrangement, operation, and details of the methods and systems disclosed.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

1. An SDD, comprising:
a non-conductive housing having a surface and that contains supporting logic circuitry;
at least one electrically conductive pad positioned on the housing surface and electrically connected to the logic circuitry and to at least one targeting agent capable of binding with a target;
signaling circuitry connected the logic circuitry; and
wherein, in response to a binding event detected by the logic circuitry, the signaling circuitry outputs a signal indicative of a magnitude and/or duration for the detected binding event.

2. The SDD of claim 1, wherein the signal comprises an analog signal that represents a voltage signal sensed by the at least one electrically conductive pad over time, wherein the voltage signal is representative of an occurrence of the detected binding event, the magnitude of the detected binding event, and the duration of the detected binding event.

3. The SDD of claim 1, wherein the signal comprises a digitization of an analog signal that represents a voltage signal sensed by the at least one electrically conductive pad over time, wherein the voltage signal is representative of an occurrence of the detected binding event, the magnitude of the detected binding event, and the duration of the detected binding event.

4. The SDD of claim 1, wherein the logic circuitry comprises one or more analog-to-digital converters (ADCs) configured to digitize one or more voltage signals from one or more of the at least one electrically conductive pad.

5. The SDD of claim 1, wherein the signal comprises a digital signal that represents a time series of binding measurement data derived from a voltage signal sensed by the at least one electrically conductive pad over time, wherein the time series of binding measurement data comprises a plurality of binding status data values with corresponding time values that indicate whether a binding event occurred and a plurality of signal magnitude data values with corresponding time values that represent magnitudes of binding events for corresponding times when binding events occurred.

6. The SDD of claim 5, wherein the logic circuitry comprises:
binding detection logic configured to process the voltage signal with respect to a threshold to generate binding occurrence data indicative of whether a binding event has occurred;
an analog-to-digital converter (ADC) configured to digitize the voltage signal to generate signal magnitude data that represents magnitude values for the binding event; and
measurement processing logic configured to correlate the binding occurrence data and the magnitude data with time based on a clock signal.

7. The SDD of claim 1, wherein the signal further indicates k_{ON}, K_{OFF}, and/or k_{D} values for the detected binding event.

8. The SDD of claim 1, further comprising:
clock circuitry that provides clock data associated with the binding event detected by the logic circuitry.

9. The SDD of claim 8, wherein the signaling circuitry outputs the signal so that the signal is correlated with time based on the clock data.

10. The SDD of claim 1, further comprising:
pad addressing circuitry;
wherein the at least one electrically conductive pad is addressable with the pad addressing circuitry.

11. The SDD of claim 1, wherein each addressable electrically conductive pad is associated with a unique pad identifier.

12. The SDD of claim 11, wherein the signaling circuitry further outputs the unique pad identifier associated with binding event data, clock data, or both.

13. The SDD of claim 1, further comprising:
a casing that at least partially encloses the SDD, the casing further comprising:
at least one port for introducing one or more targets to the at least one targeting agent.

14. The SDD of claim 1, wherein the signaling circuitry outputs the signal wirelessly to a user device and/or over a wire to the user device.

15. The SDD of claim 1, further comprising power circuitry connected to the signaling circuitry and the logic circuitry.

16. The SDD of claim 15, wherein the power circuitry powers the SDD from an RF induction source, a DC power source, and/or an AC power source.

17. The SDD of claim 1, wherein the signaling circuitry is further configured to receive input signals from a user device.

18. The SDD of claim 1, further comprising:
a modulator for encoding binding event data representative of the magnitude and duration of the detected binding event within the signal for output to the user device.

19. The SDD of claim 1, further comprising:
at least one electrically conductive needle extending from the housing, with the needle electrically connected to the logic circuitry and to a needle driving circuit.

20. The SDD of claim 19, wherein the at least one electrically conductive needle electroporates the target in response to a binding event detected by the logic circuitry.

21. A method of measuring binding event-related data, comprising:
supplying power to an SDD, the SDD having a non-conductive housing having a surface and that contains supporting logic circuitry and at least one electrically conductive pad positioned on the housing surface, with the at least one electrically conductive pad connected to the logic circuitry and to at least one targeting agent capable of binding with a target;
exposing the at least one targeting agent to a biological sample of interest;
receiving at a user device, via signaling circuitry associated with the SDD, binding data associated with the at least one electrically conductive pad.

22. The method of claim 21, further comprising:
processing the binding data at the user device to calculate binding affinity, occupation time, or both; and
displaying the processed binding data.
